SEQUENCE LISTING (18 sequences)

SEQ ID NO: 1 - protein, 2440 aa, Homo sapiens
MAENLLDGPPNPKRAKLSSPGFSANSNTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSSPSLPKQAASTSGPTPAASQALNPQAQKQVGLATSSPATSQTGPGICMNANFNQTHPGLLNSNSGHSLINQASQGQAQVMNGSLGAAGRGRGAGMPYTAPAMQGASSSVLAETLTQVSPQTAGHAGLNTAQAGGMAKIGMNGTTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQSVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSTVASAETNSQQPGPDVPVLEMKTETQAEDTEPDPGESKGEPRSEMMEEDLQGASQVKEETDIAEQKSEPMEVEDKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQDTALIAPHQIQGCVYISYLDSIHFFRPRCLRTAVYHEILIGYLEYVKKLVYVTAHIWACPPSEGDDYIFHCHPPDQKIPKPKRLQEWYKKMLDKAFAERIINDYKDIFKQANEDRLTSAKELPYFEGDFWPNVLEESIKELEQEEEERKKEESTAASETPEGSQGDSKNAKKKNNKKTNKNKSSISRANKKKPSMPNVSNDLSQKLYATMEKHKEVFFVIHLHAGPVISTQPPIVDPDPLLSCDLMDGRDAFLTLARDKHWEFSSLRRSKWSTLCMLVELHTQGQDRFVYTCNECKHHVETRWHCTVCEDYDLCINCYNTKSHTHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHNVRQQQIQHCLQQAQLMRRRMATMNTRNVPQQSLPSPTSAPPGTPTQQPSTPQTPQPPAQPQPSPVNMSPAGFPNVARTQPPTIVSAGKPTNQVPAPPPPAQPPPAAVEAARQIEREAQQQQHLYRANINNGMPPGRDGMGTPGSQMTPVGLNVPRPNQVSGPVMSSMPPGQWQQAPIPQQQPMPGMPRPVMSMQAQAAVAGPRMPNVQPNRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPPQPAMGGLNPQGQALNIMNPGHNPNMTNMNPQYREMVRRQLLQHQQQQQQQQQQQQQQQNSASLAGGMAGHSQFQQPQGPGGYAPAMQQQRMQQHLPIQGSSMGQMAAPMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGTPHPGLAVTMASSMDQGHLGNPEQSAMLPQLNTPNRSALSSELSLVGDTTGDTLEKFVEGL

SEQ ID NO: 2 - protein, 2414 aa, Homo sapiens
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGTNAGMNPGMLAAGNGQGIMPNQVMNGSIGAGRGRQDMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSLGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPNAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPTAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQPQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 3 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 4 - protein, 745 aa, Homo sapiens
MEVMNLMEQPIKVTEWQQTYTYDSGIHSGANTCVPSVSSKGIMEEDEACGRQYTLKKTTTYTQGVPPSQGDLEYQMSTTARAKRVREAMCPGVSGEDSSLLLATQVEGQATNLQRLAEPSQLLKSAIVHLINYQDDAELATRALPELTKLLNDEDPVVVTKAAMIVNQLSKKEASRRALMGSPQLVAAVVRTMQNTSDLDTARCTTSILHNLSHHREGLLAIFKSGGIPALVRMLSSPVESVLFYAITTLHNLLLYQEGAKMAVRLADGLQKMVPLLNKNNPKFLAITTDCLQLLAYGNQESKLIILANGGPQALVQIMRNYSYEKLLWTTSRVLKVLSVCPSNKPAIVEAGGMQALGKHLTSNSPRLVQNCLWTLRNLSDVATKQEGLESVLKILVNQLSVDDVNVLTCATGTLSNLTCNNSKNKTLVTQNSGVEALIHAILRAGDKDDITEPAVCALRHLTSRHPEAEMAQNSVRLNYGIPAIVKLLNQPNQWPLVKATIGLIRNLALCPANHAPLQEAAVIPRLVQLLVKAHQDAQRHVAAGTQQPYTDGVRMEEIVEGCTGALHILARDPMNRMEIFRLNTIPLFVQLLYSSVENIQRVAAGVLCELAQDKEAADAIDAEGASAPLMELLHSRNEGTATYAAAVLFRISEDKNPDYRKRVSVELTNSLFKHDPAAWEAAQSMIPINEPYGDDMDATYRPMYSSDVPLDPLEMHMDMDGDYPIDTYSDGLRPPYPTADHMLA

SEQ ID NO: 5 - DNA, 24 bp, Artificial Sequence
  Oligonucleotide
agggtctgctactgagatgctctg

SEQ ID NO: 6 - DNA, 24 bp, Artificial Sequence
  Oligonucleotide
caaccactggtttttctgccaccg

SEQ ID NO: 7 - DNA, 22 bp, Artificial Sequence
  Oligonucleotide
ggtgaaggtcggtgtgaacgga

SEQ ID NO: 8 - DNA, 22 bp, Artificial Sequence
  Oligonucleotide
tgttagtggggtctcgctcctg

SEQ ID NO: 9 - DNA, 24 bp, Artificial Sequence
  Oligonucleotide
ggcgttctctttggaaaggtgttc

SEQ ID NO: 10 - DNA, 20 bp, Artificial Sequence
  Oligonucleotide
ctcgaaccacatccttctct

SEQ ID NO: 11 - DNA, 20 bp, Artificial Sequence
  Oligonucleotide
gtcttatcgatgctggagtg

SEQ ID NO: 12 - DNA, 20 bp, Artificial Sequence
  Oligonucleotide
aaagctcttctcgcagccat

SEQ ID NO: 13 - DNA, 20 bp, Artificial Sequence
  Oligonucleotide
gcatgtcctactcgcagcag

SEQ ID NO: 14 - DNA, 20 bp, Artificial Sequence
  Oligonucleotide
gctgatcatgtcccggaggt

SEQ ID NO: 15 - DNA, 21 bp, Artificial Sequence
  Oligonucleotide
accaacagcaactatgacctc

SEQ ID NO: 16 - DNA, 20 bp, Artificial Sequence
  Oligonucleotide
aaggacgtagcgaccgcaac

SEQ ID NO: 17 - DNA, 21 bp, Artificial Sequence
  Oligonucleotide
tgcttatggatcccagagtga

SEQ ID NO: 18 - DNA, 21 bp, Artificial Sequence
  Oligonucleotide
ttggtgaggatctctccgcgt

(19) United States
(12) Patent Application Publication
(10) Pub. No.: US 2007/0128669 A1
(43) Pub. Date: Jun. 7, 2007

(54) Serum-free expansion of cells in culture

(75) Inventor: Michael Kahn, Los Angeles, CA (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Institute for Chemical Genomics, Kirkland, WA (US)

(21) Appl. No.: 11/605,202

(22) Filed: Nov. 27, 2006

Related U.S. Application Data
(60) Provisional application No. 60/740,173, filed on Nov. 28, 2005.

(51) Int. Cl. G01N 33/567; G01N 33/53

(52) U.S. Cl. 435/7.2

(57) ABSTRACT

Methods and agents are disclosed for modulating the interaction of β-catenin or γ-catenin with CBP or p300. Agents that increase the binding of CBP to β-catenin are associated with enhancing the β-catenin-related proliferation of adult stem cells, including hematopoietic stem cells, neural stem cells, skin stem cells, and pancreatic stem cells, as well as embryonic stem cells.

### CROSS REFERENCE TO RELATED APPLICATIONS

This application claims the benefit of U.S. Provisional Application No. 60/740,173, filed Nov. 28, 2005, which is incorporated herein by reference in its entirety.

### FIELD OF THE INVENTION

The present invention relates to compounds and methods for modulating the interaction between β-catenin or γ-catenin and the coactivator protein CBP, or β-catenin or γ-catenin and the coactivator protein p300, to promote proliferation/dedifferentiation or differentiation of stem/progenitor cells.

### DESCRIPTION OF THE RELATED ART

Stem cells have received significant interest over the last few years due to their potential, under suitable cellular microenvironments, to differentiate and develop into a wide array of cell and tissue types. Several important biomedical applications would be enabled by the ability to generate sufficient pools of adult stem cells, including cell replacement therapy, gene therapy, and tissue engineering. According to the National Institutes of Health, the therapeutic use of stem cells will become a cornerstone of medicine within the next two decades:
Given the enormous potential of stem cells to the development of new therapies for the most devastating diseases, when a readily available source of stem cells is identified, it is not too unrealistic to say that this research will revolutionize the practice of medicine and improve the quality and length of life (National Institutes of Health. Stem Cells: Scientific Progress and Future Research Directions. Jun. 17, 2001).

However, the development of such applications for adult stem cells has been severely impaired due to the inability to propagate and expand functional adult stem cells in culture. To date, this has proven to be a singular challenge in stem cell research (Sherley, J. (2002) Stem Cells, 20:561-572). For decades, scientists have attempted to grow stem cells in culture to increase the number of cells for transplantation. The challenge of this undertaking lies in the stem cell's predisposition to differentiate. This problem may be associated with the inherent asymmetric cell kinetics of stem cells in postnatal somatic tissues (Sherley, J. (2002) Stem Cells, 20:561-572). Existing scientific methods used for increasing the number of stem cells include culturing cells on 2-D stromal layers and growing them in the presence of various cytokine cocktails (Rebel, V I., et al. (1994) Blood, 83(1):128-136). However, none of the existing ex vivo methods can prevent differentiation of stem cells while promoting proliferation (Rebel, VI. et al. (1996)J Hematother, 5(1):25-37). There is therefore a need in the art for compounds and methods for use in propagating and expanding adult stem cells in culture.

### BRIEF SUMMARY OF THE INVENTION

The invention relates to compounds and methods for modulating the interaction between β-catenin (or γ-catenin) and the coactivator proteins CBP and p300 to either promote proliferation/dedifferentiation or differentiation of stem/progenitor cells.

In an embodiment of this method, the agent increases the binding of β-catenin to CBP. In a further embodiment of the method, the agent decreases the binding of p300 to β-catenin.

In embodiments of the method, the agent increases the binding of p300 to β-catenin, or the agent decreases the binding of CBP to β-catenin.

The cell may be treated with the agent of the invention ex vivo and the cell may be a stem cell/progenitor cell.

In certain embodiments, the agent is applied topically to a mammal comprising said cell.

In other specific embodiments, the agent modulates the interaction of Ser 89 phosphorylated p300 with a 14-3-3 protein, and the agent may be an analog of Fusicoccin, wherein the analog of Fusicoccin has the following general formula:

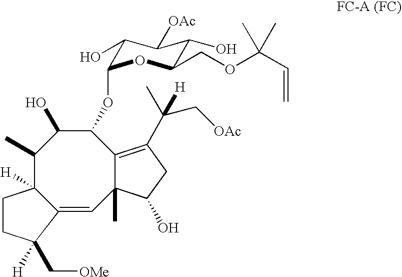

The invention also relates to a method of modulating the interaction of β-catenin with CBP or p300 in a cell, wherein the agent modulates the interaction of prolyl isomerase (Pin1) with β-catenin, CBP or p300; in certain embodiments, the agent increases the association of Pin1 with CBP.

In all these embodiments, the agent may be incorporated into a biomaterial capable of supporting the growth of a stem cell; the stem cell may be a hematopoietic stem cell.

The invention further relates to a method of enhancing the proliferation of a mammalian stem cell, comprising modulation of the interaction of β-catenin with CBP or p300; the agent may increase the binding of β-catenin to CBP; and the agent may decrease the binding of β-catenin to p300.

The administration to the stem cell may be ex vivo, and the stem cells may be hematopoietic stem cells, hair cells, neural stem cells, pancreatic islet cells, or embryonic stem cells.

The invention also relates to a method of maintaining embryonic stem cells in an undifferentiated state, comprising administering to the stem cell an agent, such as IQ-1, that selectively interacts with a differentially spliced regulatory subunit of PR72/130 of the serine/threonine protein phosphatase PP2A, where in the interaction of β-catenin/CBP is increased.

The invention further relates to a method of maintaining embryonic stem cells in an undifferentiated state, comprising administering to the stem cell an agent that selectively interacts with a differentially spliced regulatory subunit of PR72/130 of the serine/threonine protein phosphatase PP2A, where in the interaction of β-catenin/p300 is decreased.

In certain embodiments of the method, the compound(s) inhibit the MAPK kinase pathway (MEK or ERK) and PKC in conjunction with stimulation of the canonical Wnt pathway, either with Wnt3a or a GSK3 inhibitor.

The invention further relates to assays for identifying compounds suitable for maintaining and/or promoting stem cell self-renewal. One such assay measures selective binding of a compound or agent to one or more of the PR72/130 subunits of the serine/threonine protein phosphatase PP2A. In certain embodiments, the agent will have activity similar to IQ-1.

### BRIEF DESCRIPTION OF THE DRAWINGS

FIG. 1. FIG. 1 Å-D shows that IQ-1 maintains undifferentiated state of ESCs. FIG. 1A, Structure of IQ-1. FIG. 1B, IQ-1 dose dependently maintains alkaline phosphatase activity. FIG. 1C, IQ-1, dose dependently, maintains SSEA-1 expression. SSEA-1 expression was analyzed 7 days after addition of IQ-1. FIG. 1D, IQ-1 enabled ESCs to proliferate in the undifferentiated state for at least 65 days, without MEF feeders or LIF. ESCs, in media supplemented with 4 μg/ml IQ-1, were passaged 2-3 times every week at 1×10⁵-1×10⁶ cells per 6 cm dish and counted. All error bars represent mean±SD.

FIG. 2. FIG. 2A-C shows that IQ-1 maintained ESCs self-renewal independently of LIF. FIG. 2A, IQ-1 increases Nanog gene expression significantly compared to LIF. mRNA was isolated from ESCs, cultured under feeder-free system, and in the presence of either IQ-1 (4 μg/ml) or LIF (1000 U/ml) for 21 hrs. Real-time RT-PCR for Nanog was performed. The control expression level of Nanog at day 0 was set at 1. FIG. 2B, removal of IQ-1 decreases Nanog gene expression in ESCs. IQ-1 was removed from ESCs which had been cultured under feeder-free system and in the presence of IQ-1 (4 μg/ml), for three days. At the end of this period, mRNA was isolated and real-time RT-PCR was performed to assay for Nanog gene expression. FIG. 2C, effects of IQ-1 were not mediated through Stat3 signaling pathway as judged by luciferase reporter assay. Feeder-free ESCs, transfected with the pSTAT3-TA-Luc reporter, were exposed to IQ-1 at the indicated doses, or LIF. All error bars represent mean±SD.

FIG. 3. FIG. 3A-C shows that IQ-1 modulates Wnt signaling via interaction with PR72/130. FIG. 3A, affinity chromatography isolation of IQ-1 's molecular target(s) were performed as described in Experimental Procedures. The two bands at 72 kDa and 130 kDa (labeled) were identified by mass spectral sequencing as the differentially spliced regulatory subunits PR72/130 of the serine/threonine protein phosphatase, PP2A. FIG. 3B, immunoblotting, using PR72/130 antisera, was performed to confirm the identity of the two bands. FIG. 3C, IQ-1 causes developmental defects in zebrafish. 1-cell stage zebrafish embryos were treated with 1 μM IQ-1 (Lower) or DMSO control (Upper) for 24 hrs. Results are representative of at least 10 embryos, from three independent experiments.

FIG. 4. FIG. 4A-D shows that IQ-1 maintenance of ESCs is Wnt/β-catenin/CBP dependent. FIG. 4A, Wnt/β-catenin Coactivator Switching Model. FIG. 4B, IQ-1 increased the CBP/β-catenin complex at the expense of the p300/β-catenin complex. P19 cells were treated with Wnt3A supplemented with IQ-1, the CBP/β-catenin ICG-001 or DMSO control. Nuclear lysates were co-immunoprecipitated with anti-CBP or anti-p300 antibody and immunoblotted for β-catenin. FIG. 4C, phosphorylation of p300 Ser89, in a PKC dependent manner, increased the p300/β-catenin interaction. After in vitro phosphorylation with PKCα, wild type p300 (1-110 aa) and the mutant p300 (p300 S89A) were mixed with P19 lysates and co-immunoprecipitated using the HA-tag. Western blot analysis for p300 (Upper) or β-catenin loading control (Lower) were performed. Lane 1, p300/β-catenin binding, Lane 2, PKCα phosphorylated p300/β-catenin binding, Lane 3, S89A p300/β-catenin binding, Lane 4, PKCα phosphorylated S89A p300/β-catenin binding. FIG. 4D, IQ-1 decreased the phosphorylation of p300. P19 cells were treated with IQ-1 or DMSO (control) and exposed to purified Wnt3A for 24 hrs. Cell lysates were immunoblotted using antibodies specific for p300, or p300 phosphorylated at position Ser 89. Lane 1 negative control, Lane 2, Wnt3a plus DMSO control, Top panel phospho Ser89 p300 immunoblot, Middle panel p300 immunoblot, Lane 3, Wnt3a plus IQ-1, Top panel phospho Ser89 p300 immunoblot, Middle panel p300 immunoblot.

FIG. 5. FIG. 5 A-C shows the pluripotency of long term cultured ESCs. FIG. 5A, long term cultured ESCs were induced to form embryoid bodies in suspension cultures for 3 days. ESCs cultured in media in the presence of Wnt3A and IQ-1 (4 μg/ml) for 48 days were able to form embryoid bodies (Left). ESCs lose their ability to form embryoid bodies after 3 days of culturing in the absence IQ-1 (Right). FIG. 5B, IQ-1 treated ESCs derived embryoid bodies were cultured (adherence culture) for 7-14 days to induce further differentiation. Immunofluorescence staining for α-fetoprotein, smooth muscle actin, GATA4, MAP2, β-III tubulin and oligodendrocytes demonstrated that long term culture of ESCs in the presence of IQ-1, preserves pluripotency. FIG. 5C, model depicting the proposed mechanism of action of IQ-1.

FIG. 6. FIG. 6 shows TCF/β-catenin reporter gene analysis. The TCF/β-catenin reporter construct TopFlash was cotransfected with a constitutively active β-catenin into NIH-3T3 cells-wt, CBP (+/−) and p300 (+/−)—in the presence or absence of IQ-1. There was no effect of IQ-1 on the Fopflash reporter.

FIG. 7. FIG. 7 shows long term culture of ESCs in feeder free system with serum-free media supplemented with IQ-1 and purified Wnt3a. ESCs were cultured in 15% KSR, 4 μg/ml of IQ-1 and 100 ng/ml Wnt3a. ESCs were passaged 2-3 times per week at 1×10⁵-1×10⁶ cells per 6 cm dish and cells were counted.

FIG. 8. FIG. 8 shows that IQ-1 maintenance of ESC proliferation and pluripotency was dependent on Wnt signaling in 15% KSR media. AP activity of ESCs cultured for 7 days in IQ-1 containing media supplemented with 15% FCS or 15% KSR. Addition of Wnt3a increases AP activity of ESCs cultured for 7 days in 15% KSR containing media to the same level as 15% FCS containing media. (Error bars represent ±S.D.)

FIG. 9. FIG. 9 shows real time RT-PCR performed on ESCs cultured in 15% KSR supplemented with IQ-1 and Wnt3a for 42 days without MEF feeders. Over the 42-day culture period, ESCs cultured in the media in the presence of IQ-1 and Wnt3a maintain expression of the pluripotency markers, (a) Nanog, (b) Oct3/4 and (c) Rex-1. The expression level on day 0 was set as 1.

FIG. 10. FIG. 10 is a schematic representation of two possible mechanisms of action of β-catenin, resulting from alternative interaction with CBP or p300 in the nucleus.

FIG. 11. FIG. 11 illustrates that ICG-001 at 10 μM concentration induced the differentiation of C2C12 myoblasts in growth medium, similar to differentiation media, and compared to growth medium alone.

FIG. 12. FIG. 12 illustrates that differentiation of C2C12 myoblasts was induced by 10 μM ICG-001 in growth medium, similar to differentiation medium.

### DETAILED DESCRIPTION OF THE INVENTION

Stem cells are responsible for the regeneration and maintenance of tissues by balancing the processes of self-renewal (i.e., making new stem cells) and differentiation (i.e., generating cells committed to terminal differentiation). This balance results from integration of regulatory signals intrinsic to the stem cell, as well as extrinsic signals from the microenvironment. Perturbations in the balance between self-renewal and differentiation may result in disease, either as a result of stem cell depletion (e.g., aplastic anemia) or increased self-renewal (e.g., cancer). In neurogenesis, the transcription factors Sox1, Sox 2 and Sox3 play a role in maintaining neural cells in an undifferentiated state.

Most knowledge about the molecular mechanisms of stem cell regulation in mammals has been derived from studies of the hematopoietic system. There is an extensive and expanding understanding of the molecular mechanisms that regulate differentiation along the terminal lineages. However, a mechanistic understanding of the mechanisms that regulate hematopoietic stem cell (HSC) fate decisions is less well understood. A few genes have been identified that, when deleted, result in perturbation of HSC self-renewal (e.g. TNFα-p55-Receptor, p21, Rae28, and Bmi-1) or altered differentiation (e.g., TEL, PU.1, Flt-3, and p27). HoxB4, β-catenin, and Notch signaling, on the other hand, stimulate HSC self-renewal when over-expressed in HSCs.

Recent work has demonstrated that CBP and p300 play important roles in HSC self-renewal and differentiation. CBP and p300 function as molecular integrators of various transcriptional signals. When recruited to promoters by transcription factors, they function as co-activators of transcription through multiple mechanisms, including chromatin remodeling, acetylation of associated proteins, and recruitment of the basal transcription machinery. CBP and p300 are highly homologous on a structural level, with up to 93% identity within certain protein-binding domains (SEQ ID NO: 1 and 2). For most functions, the two proteins appear to be functionally redundant. However, mouse genetic loss-of-function studies demonstrated a difference between p300 and CBP function in HSCs: loss of CBP results in defective HSC self-renewal, whereas loss of p300 results in defective hematopoietic differentiation.

CBP and p300 have been previously shown to interact with many of the known transcription factors shown to be important in HSC regulation (e.g., HoxB4, β-catenin, Notch, AML-1, MLL). Earlier results suggest that within HSCs there may be transcription factors that are specifically co-activated by CBP that are critical for self-renewal, and others that are preferentially co-activated by p300 that are critically required for differentiation. One example of a signaling pathway that seems to utilize CBP and p300 differentially is the Wnt signaling pathway. The Wnt signaling pathway has been shown to play a pertinent role in the development and maintenance of various tissues, including blood, intestines, and skin. Its effects are executed at the level of stem and progenitor cells, affecting both self-renewal and differentiation. Moreover, the importance of Wnt signaling in maintaining the undifferentiated features of embryonic stem (ES) cells has been well established. Importantly, when Wnt signaling is perturbed it can lead to the development of cancer in these same tissues.

β-catenin (SEQ ID NO:3) is a vertebrate homolog of Drosophila gene armadillo, which functions in both cell adhesion and, as discussed herein, the Wnt signaling pathway. γ-catenin (SEQ ID NO:4) is also a vertebrate homolog of armadillo. β-catenin and γ-catenin have analogous structures and functions, and they have the ability to be regulated by the APC tumor suppressor.

Activation of the Wnt signaling pathway requires the nuclear stabilization of TCF (T cell factor)/β-catenin complexes and recruitment of transcriptional co-activators, such as CBP and p300. β-catenin is constitutively produced in the cell, and inhibitory mechanisms exist to maintain β-catenin levels at below those that would lead to aberrant transcriptional activity in vivo, leading to pathological conditions such as cancer. In one example of aberrant regulation, Emami and colleagues (PNAS, 101, 12682-7, 2004) recently demonstrated that β-catenin preferentially associates with CBP in cancer cells. However, when β-catenin was prevented from associating with CBP, by utilizing a β-catenin/CBP-specific inhibitor, β-catenin could bind to p300. The “alternative” binding of β-catenin to p300 was accompanied by the execution of a differentiative genetic program (Teo et al. PNAS submitted). Thus, β-catenin is thought to promote proliferation without differentiation by binding to and activating CBP, and to initiate differentiation with limited proliferation by binding to and activating p300. Perturbation of β-catenin interation with CBP and/or p300 is expected therefore to influence differentiation or proliferation.

Stem cell therapy is based on the ability of human fetal or adult pluripotent stem cells to differentiate into a variety of cell types. Stem cells may be used to replace damaged cells as a treatment for many different diseases including cancer, Parkinson's disease, spinal cord injury, burns, diabetes, heart disease, rheumatoid arthritis, and osteoarthritis and for gene therapy (Lazic, S. E. et al. J Hematother Stem Cell Res, 12(6):635-642, Gafni, Y. et al. Gene Ther, 11(4):417-426). Stem cell therapy has long been an exciting potential medical breakthrough. The ability to inject normal stem cells into a patient, where they could generate organ-specific cells to potentially replace defective patient tissues, offers enormous potential.

Embryonic stem cells (ES cells) represent an important research tool and a potential resource for regenerative medicine. Generally, ES cells are cocultured with a supportive feeder cell layer of murine embryonic fibroblasts (MEFs). The MEF feeder layer supplies factors which maintain the ES cell's capacity for self renewal and block spontaneous differentiation. These cumbersome conditions, as well as the risk of xenobiotic contamination of human ES cells grown on MEFs, make it a priority to develop chemically defined media that can be safely utilized for the expansion of ES cells. Using a high throughput cell based screen, the small molecule IQ-1 was identified as a compound that allowed for the expansion of mouse ES cells without a MEF feeder layer or the addition of leukemia inhibitory factor (LIF), and prevented spontaneous differentiation. It has also been determined that IQ-1 prevents β-catenin from switching coactivator usage from CBP to p300. The increase in β-catenin/CBP mediated transcription at the expense of β-catenin/p300 mediated transcription is critical for the maintenance of stem cell pluripotency.

ES cells derived from the inner cell mass of embryos can be cocultured on embryonic fibroblast cell layers. More recently, Xu et al. (Xu et al. Nat. Biotech. 2001, 19, 971) demonstrated that human ES cells can be grown under feeder free conditions utilizing MEF conditioned media. However variations in MEFs and MEF conditioned media, the lack of knowledge about what factors in conditioned media are important and concerns about zoonotic contamination emphasize the need for chemically defined conditions to expand ES cells.

The ability to maintain adult skin stem cells in vitro has allowed engraftment of cultured skin onto burn victims (Green, H. (1991) Sci Am, 265:96-102). Additionally, at present, there are three adult stem cell related transplantation procedures used for hematopoietic reconstititution: bone marrow transplantation (BMT), peripheral blood stem cell transplantation (PBSCT) and umbilical cord blood stem cell transplantation (UCBSCT). The first two hematopoietic reconstitution techniques, BMT and PBSCT, suffer from a significant matching problem with allogeneic donors. The degree of match required for a successful transplant appears to be less stringent for UCBSCT than BMT or PBSCT. However, the relatively lower volume of harvested stem cells and the availability of only one collected cord blood unit per transplant procedure limit the wide applicability of UCBSCT (McCaffrey, P. Lancet Oncol., 6 (1): 5, 2005). One solution to this problem is ex vivo expansion of the cord blood stem cells. However, there is a significant hurdle to overcome in order to provide this straightforward solution.

Stem Cells and Cancer “Stem Cells”

A unifying feature of all cancers is their capacity for unlimited self-renewal, which is also a defining characteristic of normal stem cells. Decades ago, it was discovered that the proliferative capacity of all cancer cells was not equivalent, and only a small minority of tumor cells were able to proliferate extensively (Hamburger, A W. et al. (1977) Science, 197(4302):461-463). This gave rise to the concept that malignant tumors are comprised of Cancer Stem Cells, which have great proliferative potential, as well as another pool of more differentiated cancer cells, with limited proliferative capacity. An important implication of the Cancer Stem Cell hypothesis is that there are mechanistic similarities between the self-renewal of normal stem cells and the proliferation of cancer stem cells (Pardal, R. et al. (2003) Nat Rev Cancer, 3(12):895-902). Recent studies have demonstrated that specific gene products regulate both the self-renewal of normal somatic stem cells, as well as the proliferation of cancer cells (Park, I K. et al. (2003) Nature 423:302-305; Lessard, J. et al. (2003) Nature, 423(6937):255-260). This implies that similar mechanisms are utilized in both stem cells and cancer cells to maintain a proliferative, non-differentiated state.

Wnt Signaling in Stem Cells and Cancer

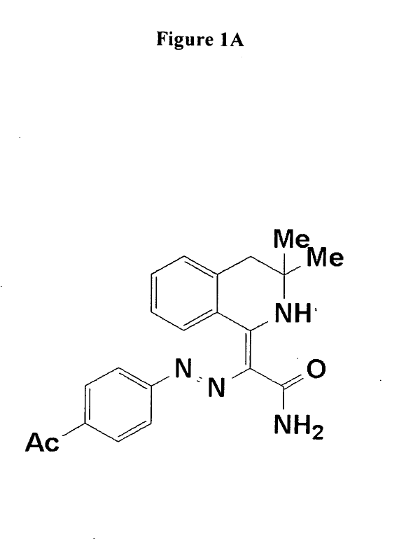

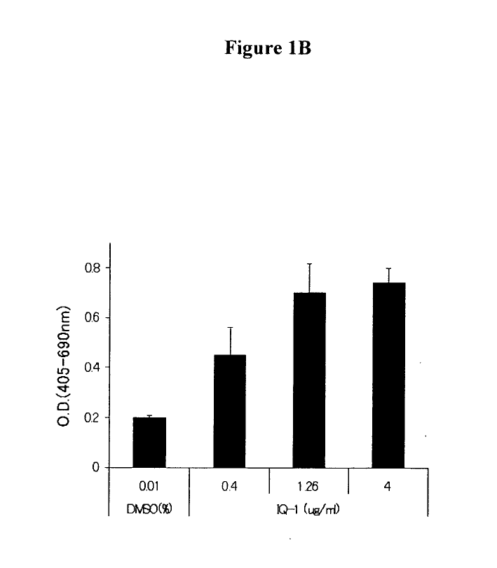

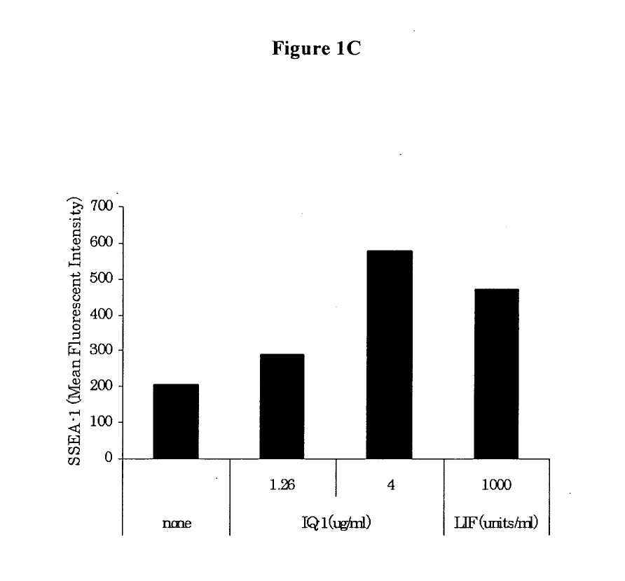

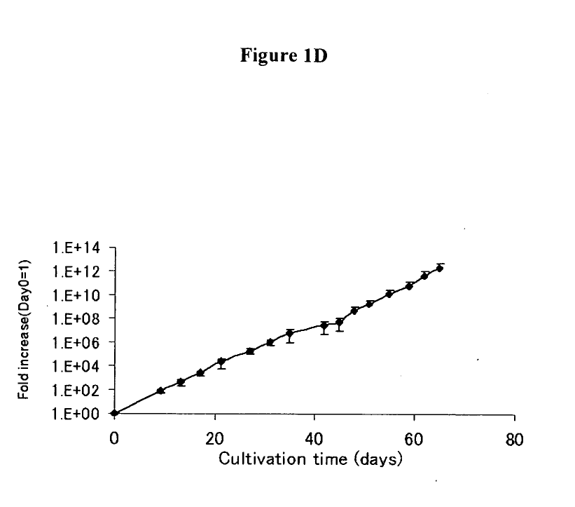

The Wnt/β-catenin pathway initiates a signaling cascade critical in both normal development and the initiation and progression of cancer (Giles, R H et al. (2003) Biochim Biophys Acta, 1653(1):1-24; Wodarz, A. et al. (1998) Annu Rev Cell Dev Biol, 14:59-88). Wnt signaling and in particular the nuclear functions of β-catenin have been shown to be important in the maintenance, proliferation as well as the differentiation of stem cells (Song, X. et al. (2003) Development, 130(14):3259-3268). Some of the salient features of this signaling pathway, relevant to this invention, are summarized in FIG. 1. The Wnt/β-catenin pathway normally regulates expression of a range of genes involved in promoting both proliferation and differentiation. Activation of the Wnt pathway allows β-catenin to accumulate in the nucleus, bind to members of the TCF family of transcription factors, and form a transcriptionally active complex, by recruiting either the transcriptional coactivator CBP or its closely related homolog, p300. However, in greater than 85% of colon cancers, mutations in this pathway lead to constitutive activation and expression of target genes, e.g. c-myc, cyclin D1 and survivin, all of which are critical for rapid cell proliferation (Kolligs, F T. et al. (1999) Mol Cell Biol, 19(8):5696-5706; Tetsu, O. et al. (1999) Nature, 398(6726):422-426; Kim, P J. et al. (2003) Lancet, 362:205-209). Thus, tumorigenesis in the intestinal epithelium appears to be caused by Wnt/β-catenin induced hyper-proliferation of intestinal crypt stem cells, followed by accumulation of additional mutations that confer malignancy and cancer progression. Wnt signaling has also been demonstrated to be important for the maintenance of pluripotency in both mouse and human embryonic stem cells in culture (Sato, N. et al. (2004) Nat Med, 10(1):55-63). Expression of multiple components of the Wnt pathway is evident in the P19 human embryonal carcinoma cell lines, as well as in embryonic stem cells (Walsh, J. et al. (2003) APMIS, 111(1):197-211).

Wnt and Hematopoietic Stem Cells (HSC)

The self-renewal of hematopoietic stem cells (HSC) is also promoted by Wnt signaling. Overexpression of stabilized β-catenin in cultured bone marrow HSC from mice increased the number of these cells in long-term culture as measured by their ability to reconstitute the hematopoietic systems of mice following irradiation. Additionally, purified Wnt3a promoted self-renewal but only partially inhibited the differentiation of HSC in culture (Reya, T. et al. (2003) Nature, 423(6938):409-414).

Differential Coactivator Usage in Wnt/β-catenin Signaling

As discussed above, the functions of CBP and p300 have been described as redundant in several studies (reviewed in Goodman, R H. et al. (2000) Genes Dev, 14(13): 1553-1577) and their expression pattern during mouse development is almost identical (Partanen, A. et al. (1999) Int J Dev Biol, 43(6):487-494). However, it is becoming increasingly clear that these highly homologous coactivators are not redundant under physiological conditions, and are responsible for distinct transcriptional programs. Rebel et al. (Rebel, V. I. et al. (2003) Proc Natl Acad Sci USA, 99(23):14789-14794), using cells from knockout mice, demonstrated that a full dose of CBP, but not p300, is crucial for HSC self-renewal. Conversely, p300 but not CBP, is essential for proper hematopoietic differentiation. Similarly, Eckner and colleagues (Roth, J. F. et al. (2003) Embo J, 22(19):5186-5196) demonstrated a critical role for p300's histone acetyltransferase activity (HAT) but not CBP's HAT activities. These studies and others clearly demonstrate that CBP and p300 play non-redundant and distinct roles during development.

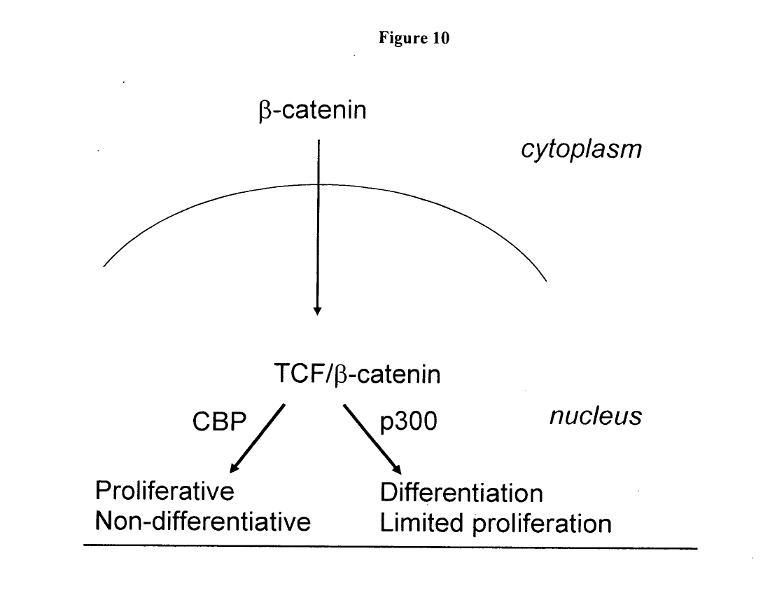

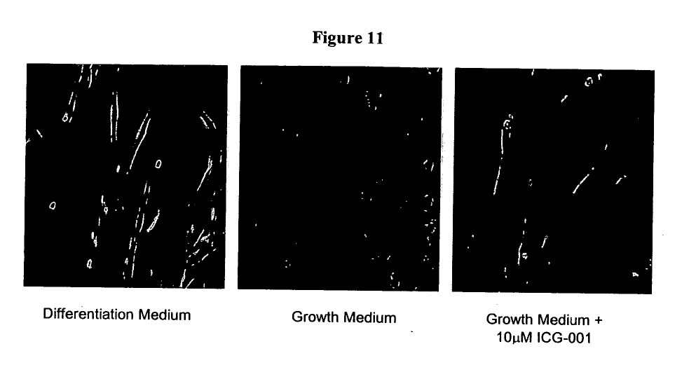

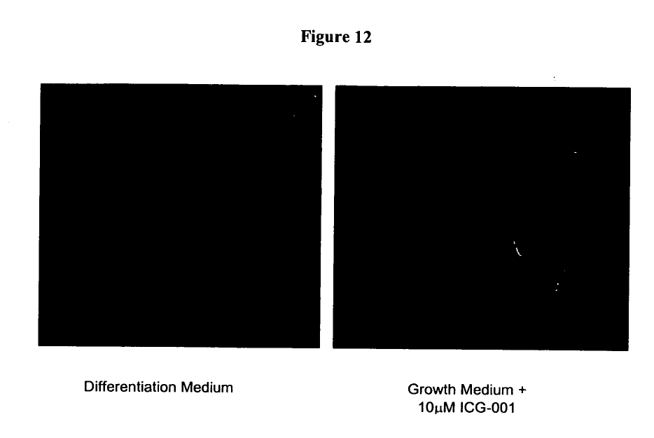

From the inventor's previous chemogenomic studies with the small molecule inhibitor of the β-catenin/CBP interaction and additional gene expression profiling, a model was developed that describes how differential coactivator usage in Wnt signaling controls proliferation vs. differentiation. The critical feature of this model is that the CBP arm (FIG. 10, left side) of the pathway is essential for proliferation without differentiation, for example in cancer or stem cells, whereas the p300 arm (FIG. 10, right side) is critical for differentiation, with limited proliferation. ICG-001 specifically inhibits β-catenin/CBP dependent transcription (i.e. the left arm of the pathway), thus selectively inducing programmed cell death in cancer cells (Emami, K. H. et al. (2003) Proc Natl Acad Sci USA, 101, 12682-7, 2004), and inducing the differentiation of non-tumorigenic precursor cells, e.g. C2C12 myoblasts (FIGS. 11 and 12) and 3T3-L1 preadipocytes.

Without being bound by a specific mechanism, the present invention is based on the premise that selectively inhibiting or down-modulating the β-catenin/p300 interaction (i.e. the right side of the pathway, FIG. 10) allows for proliferation without differentiation of pluripotent stem cells. The invention is also based in part on the discovery that one mechanism involves the selective binding of a compound or agent to one or more of the PR72/130 subunits of the serine/threonine protein phosphatase PP2A, as disclosed in more detail in Example 1 herein. In certain embodiments, the agent will have activity similar to IQ-1.

The serine/threonine phosphatase PP2A is involved in regulating intracellular signaling, gene expression and cell cycle progression. A major function of PP2A is to regulate signaling cascades by opposing the activity of serine/threonine kinases (20). PP2A consists of a multisubunit complex. The core components of this trimeric complex are a 36 kDa catalytic, a 65 kDa regulatory (PR65) and a third variable subunit, one of which is PR72/130. PR72/130 represents tissue selective differentially spliced forms of the same gene (21). PP2A regulates the Wnt signaling cascade at multiple levels (22, 23). Recently, PR72/130 has been shown to interact with the protein Naked cuticle (Nkd), a negative regulatory component of the Wnt signaling pathway (24), thereby modulating Wnt signaling (25). Creyghton et al., using morpholinos in xenopus embryos, demonstrated that PR72 like Nkd, is a “negative” regulator of “canonical” Wnt/β-catenin signaling and is involved in the switch from “canonical” Wnt signaling to “non-canonical” convergent extension (25).

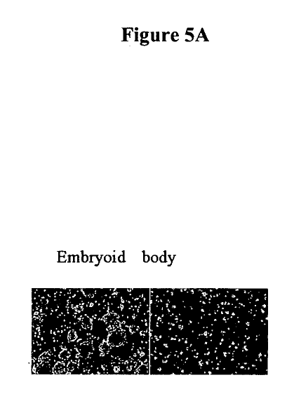

For maintenance of hematopoietic stem cell proliferation, a preferable agent of the invention “modulates” the proliferation of stem cells by affecting the post-translational modifications of any one of CBP, p300, or β-catenin, leading to a selective increase of β-catenin interaction with CBP or a selective decrease of β-catenin interaction with p300, wherein the agent does not directly bind to CBP or p300. By “interact” and “interaction” is meant the normal biological relationship between two or more molecules, in this case β-catenin with CBP or p300. In one embodiment, the agent increases the binding of β-catenin to CBP. In another embodiment, the agent decreases the binding of β-catenin to p300. With either of these embodiments, the overall result biases the β-catenin pathway towards “proliferative/non-differentiative program” of the target cells, which according to the invention are adult stem cells, such as hematopoietic stem cells, neural stem cells, or skin stem cells. An agent will “modulate” the proliferation of stem cells if the stem cells undergo more proliferation and/or less differentiation that in the absence of the agent. For example, with reference to FIG. 5C, preferential binding of β-catenin to CBP, with less binding to p300, is associated with maintaining hematopoietic stem cells in an undifferentiated state wherein they undergo continuous proliferation, resulting in enhanced numbers of undifferentiated cells useful for repopulating the hematopoietic system of a mammal, such as a human, in need of such treatment. Such modulation can be measured using, for example, assays described in the Examples herein.

Agents suitable for use according to the invention can be screened using co-immunoprecipitation methods as described in Emami et al. PNAS, 101, 12682-7, 2004. Briefly, target cells, in this case HSC, are transfected with full-length β-catenin or with full-length p300. Nuclear lysates are treated with a radiolabeled test agent alone, or with cold test agent. Unbound radiolabeled test agent is removed, and incorporation of the radiolabeled test agent is measured. The results indicate whether the test agent specifically interacts with p300.

A separate series of experiments can demonstrate inhibition of the interaction of β-catenin with p300. The minimal binding domain of CBP (amino acids 1-111), p300 (amino acids 1-111) and the C-terminal region of β-catenin (SEQ ID NO:3) (amino acids 647-781) are expressed in mammalian cells treated with the appropriate agents to modify the interaction and purified. β-catenin is bound to protein A-agarose beads coated with β-catenin-specific antibody and incubated with either CBP or p300. Unbound proteins are removed by washing, then the specific interactions between β-catenin and p300, and β-catenin and CBP, are challenged using the test agent, for testing the compounds which directly bind to CBP or phosphor Ser89 p300. Agents that either increase the binding of β-catenin to CBP or decrease the binding of β-catenin to p300 are further tested in vitro using a suitable model of hematopoietic stem cell proliferation/differentiation. One such model is described in Rebel, V. I. et al., PNAS 99:14789-14794, 2002.

Agents according to the invention may achieve the desired biological effects through one of several mechanisms. In each case, reference to “increase” or “decrease” refers to the assay results or biological effects relative to the values in the absence of the agent. For example, the agent may increase the binding of β-catenin to the amino-terminal 110 amino acids of CBP, or it may decrease the binding of β-catenin to the amino-terminal 110 amino acids of p300. The decrease in binding of β-catenin to p300 may be achieved by inhibiting the phosphorylation of Ser 89 of p300, wherein the phosphorylation is catalyzed by protein kinase C-epsilon (PKC), calcium/calmodulin-dependent protein kinase (CaMK), protease-activated receptor-4 (PAR-4), protease-activated receptor-1 (PAR-1), or other serine/threonine protein kinase either directly or indirectly via a kinase cascade.

The decreased phosphorylation of Ser 89 of p300 may be achieved by increasing the phosphorylation of Ser 90, for example by mitogen-activated protein kinase 4 (MAPK), cyclin-dependent kinase (CDK), or other serine/threonine protein kinase.

A preferable agent of the invention may modulate the interaction of Ser 89-phosphorylated p300 with 14-3-3 proteins. Such agents may be analogs of Fusicoccin. Fusicoccin is a fungal toxin that is used to study H+-ATPase activation. The mechanism involves inducing an irreversible bond between the C-terminal portion of H+-ATPase, and 14-3-3 protein. (Svennilid, F. et al., Plant Cell 11:2379-2392, 1999.) As a result, the C-terminal auto-inhibiting domain is displaced. Similarly, analogs of Fusicoccin may modulate the interaction of Ser-89 phosphorylated p300 with 14-3-3 proteins, resulting in the decreased interaction of p300 with β-catenin.

In other embodiments, the agent modulates the interaction of Pin1 with β-catenin or with CBP or p300. In one embodiment of the invention, the agent increases the association of Pin1 with β-Catenin/CBP. Pin1 (prolyl isomerase) has been implicated in cancer mechanisms by inhibiting the interaction of β-catenin with the tumor suppressor APC. Pin1 overexpression has been reported to occur in human breast cancer. (Ryo, A. et al., Nat. Cell Biol. 3:793-801 (2001)). Pin1 has also been implicated in normal spermatogenesis. Atchison, F. W. et al. (Biol. Reprod. 69:1989-1997, 2003) reported that adult Pin1-deficient mice exhibited evidence of accelerated exhaustion of stem cell potential, and possible bias towards the differentiation pathway in the absence of Pin1.

Phosphorylation affects the conformation of proteins and creates conditions for binding of signal transducers to certain suitable domains capable of recognizing the phosphorylated residue or residues. Pin1 specifically recognizes phosphorylated S/T-P bonds (Ser/Thr-Pro motifs). For example, Pin1 directly binds a phosphorylated Ser-Pro motif (Ser 246-Pro) next to the APC-binding site in β-catenin, inhibits β-catenin interaction with adenomatous polyposis coli protein (APC), and thereby increases its translocation into the nucleus. (Ryo, A. et al., Nature Cell Biol. 3:793-801, 2001.)

Pin1 can also affect coactivator interactions with transcription factors. P73 is a transcription factor related to the tumor suppressor p53. Pin 1-modified p73 displayed a higher affinity for p300 than unmodified p73. (Montovani, F. et al., Mol. Cell. 14:625-636, 2004.) Similarly, Pin1 binding to phosphorylated β-catenin can increase the β-catenin/CBP interaction and thereby β-catenin/CBP dependent gene transcription promoting proliferation at the expense of differentiation.

The agents of the invention can be incorporated into biomaterials on which hematopoietic stem cells are grown. Examples are disclosed in Horak et al., Biomaterials 25, 5249-60, 2004 and Harrison et al. Biomaterials 25, 4977-86, 2004.

Although hematopoietic stem cells are disclosed herein as an embodiment of a target for the methods of the invention, the methods are applicable to any adult mammalian stem cells (or ES cells) that can be used for tissue regeneration. Adult stem cells constitute an undifferentiated population of cells that retain the ability to proliferate throughout postnatal life and to differentiate into specialized cells to replace cells that become diseased, die or are lost. (Agrawal, S. et al; Trends in Biotechnology 23:78-83, 2005.) In addition to HSC, stem cells suitable for use according to the invention include neural stem cells, skin stem cells, muscle stem cells, and pancreatic islet cells.

The goal of diabetes treatment is to restore normal numbers and function of insulin-producing β cells. Trucco, M. (J. Clin. Invest. 115:5-12, 2005) discusses the existence of adult pancreatic precursor cells that can generate β cells, and are referred to as pancreas-derived multipotent precursors. Other stem cells may be induced to direct their differentiation toward the β cell. For either of these sources of β cells, the methods and agents of the invention are suitable for inducing proliferation and limiting differentiation, in order to achieve a suitable number of cells for therapeutic use.

Adult neural stem cells can differentiate into neurons, astrocytes, and oligodendrocytes, which are the three major lineages of the adult nervous system. For such applications of the invention, it may be appropriate to manipulate adult neural stem cells in situ in order to achieve neurogeneration in vivo. Active stem cells exist in adult brain in the dentate gyrus region of the hippocampus and the subventricular zone of the forebrain, and these stem cells can differentiate into neurons, astrocytes and oligodendrocytes. In addition, quiescent stem cell pools exist in the spinal cord, substantia nigra, optic nerve, and hypothalamus. (Agrawal, S. et al., 2005). Thus, defined pools of neural stem cells are available for modulation according to the invention.

Skin stem cells may be induced to proliferate in vivo in order to enhance or restore hair growth. Recent evidence suggests that the Wnt pathway is involved in the ability of skin epithelial cells to acquire and/or maintain characteristics of multipotent stem cells. (Alonso, L. et al.; PNAS 100:11830-11835, 2003). Multipotent stem cells in skin receive Wnt signals before they commit to form hair follicles. In transgenic mouse skin in which β-catenin is constitutively stabilized, adult interfollicular epidermis takes on characteristics of embryonic skin, and may have the capacity to develop into hair follicles. (Gat, V., Cell 95:605-614, 1998). Thus, agents and methods of the invention are suitable for enhancing the proliferation of multipotent stem cells in the skin, to provide a reservoir of cells capable of forming hair follicles in order to increase or replace lost hair growth, including in vive applications, for example by topical use. U.S. Pat. No. 6,419,913 discloses compositions suitable for topical delivery of therapeutic agents including agents for treatment of hair loss. U.S. Pat. No. 6,680,344 also discloses topical delivery of agents for treating hair loss.

In addition to using stem cells following proliferation induced by the agents and methods of the invention, it is also feasible to alter the stem cells prior to use, by gene therapy. The invention therefore provides methods to enhance the proliferation of mammalian stem cells expressing an exogenous gene, prior to administration of the cells for therapeutic use. The gene therapy may also be conducted in vivo, for example, to alter the differentiation potential of neural stem cells. (Gomes, W. A. et al., Dev. Biol. 255:164-177, 2003; Pardridge, W. M., Curr. Opin. Drug Discov. Devel. 6:683-691, 2003.)

An assay suitable for determining whether mammalian stem cells are maintained in a non-differentiated state involves the use of a reporter gene under the control of the OCT4 promoter. OCT4 is a known marker of the undifferentiated stem/progenitor cell state, and the promoter region can be functionally linked to a reporter gene such as EGFP (enhanced green fluorescent protein) as described in Gerrard, L. et al., Stem Cells 23:124-133 (2005)), or luciferase. Using either reporter gene, cells are transfected with an OCT4-reporter gene construct using methods described in Gerrard et al. (2005) and the effect of agents according to the invention on the undifferentiated versus differentiation state of the cells is tested.

Methods for testing the effect of small molecules on stem cells in vitro include those described by Chen, J. K. et al., P.N.A.S. 99:14701-14076 (2002) and Frank-Kamenetsky, M. et al., J. Biol 1:10 (2002).

Embryonic stem cells represent an important tool for research and in principle, a potential resource for regenerative medicine (Hori Y et al Proc. Natl. Acad. Sci. USA 2002, 99, 16105, Kim J H et al. Nature, 2002, 418, 50, Lanza R P et al, Nat Med, 1999, 5, 975). Murine ES (mES) cells, derived from the pluripotent inner cell mass, can be grown in the absence of feeder cells in media supplemented with serum and LIF. The ability of LIF to maintain mES cell pluripotency, requires activation of the STAT3 signaling pathway. However, LIF does not maintain the undifferentiated state of hES cells despite the fact that it activates the STAT3 signaling pathway (Humphrey R and Beattie G Stem Cells 2004, Daheron L and Opitz S Stem Cells 2004).

As shown in detail in the Examples, IQ-1 dose dependently maintained ES cell proliferation and pluripotency. IQ-1 could maintain ES cell proliferation in the absence of serum if the media was supplemented with Wnt3a. Importantly, Wnt3a plus IQ-1 was sufficient to maintain ES cell proliferation and pluripotency for extended periods of time in culture (at least for 48 days). Wnt3a plus IQ-1 increased the expression of Oct4 and Sox2 and decreased the expression of c-myc in P19 embryonic carcinoma cells. Recently, Boyer et al. (Cell 122, 947, 2005) demonstrated that Oct4 and Sox2 co-occupy the promoters/enhancers of a substantial portion of the genes required for the maintenance of human ES cells. Furthermore, c-myc appears to be a critical player in the balance between stem cell self renewal and differentiation and increased upon differentiation (Wilson A et al Genes Dev 18, 2747, 2004).

Canonical or Wnt/β-catenin signaling plays a crucial role in regulating the expansion of both human and mouse stem cell populations (Kleber M and Sommer L Current Op Cell Bio 2004, Willert K and Brown J Nature 2003, and Sato N and Meijer L Nat. Med 2004). However, numerous studies have demonstrated that Wnts can act as either a growth factor maintaining pluripotency or alternatively induce differentiation and influence cell lineage decisions (Kleber and Sommer 2004, Ille F and Sommer L Cell Mol Life Sci 2005, Murashov A, Pak E Faseb J 2004, Feng Z and Srivastava A BBRC 2004).

Recently, the inventors developed a model to explain these divergent Wnt/β-catenin signaling activities (FIG. 5). This model highlights the distinct roles of the coactivators CBP and p300 in the Wnt/β-catenin signaling pathway (Emami et al 2004, Ma et al 2005, Teo et al 2005, McMillan and Kahn 2005). The critical feature of the model is that TCF/β-catenin/CBP mediated transcription is critical for stem cell/progenitor cell proliferation, whereas a switch to TCF/β-catenin/p300 mediated transcription, whether induced chemogenomically with ICG-001 or endogenously, is critical to initiate a differentiative program with a more limited proliferative capacity. Based on this model, experiments were performed to determine whether IQ-1 affected selective CBP coactivator usage in the Wnt/β-catenin signaling pathway.

IQ-1 selectively promoted the β-catenin/CBP interaction at the expense of the corresponding p300/β-catenin interaction. Using an affinity chromatography approach, it is shown herein that the molecular targets of IQ-1 are the differentially spliced regulatory subunits PR72/PR130 of the protein phosphatase PP2A (Bernards R 2004 and in press). Bernards has previously demonstrated that PR72 interacted with the protein Nkd, a Wnt-inducible antagonist of Wnt/β-catenin signaling, and that loss of either PR72 or Nkd resulted in activation of Wnt/β-catenin signaling. IQ-1 had dramatic effects on zebrafish embryonic development and convergent extension. By identifying PR72/130 as molecular targets of IQ-1, the invention provides an assay for identifying other agents useful in promoting and/or maintaining stem cell proliferation.

The ability to expand “stem/progenitor” populations under defined growth conditions has important ramifications in the area of regenerative medicine (Hori Y et al Proc. Natl. Acad. Sci. USA 2002, 99, 16105, Kim J H et al. Nature, 2002, 418, 50, Lanza R P et al, Nat Med, 1999, 5, 975). The Wnt signaling pathway clearly plays an important role in the expansion of “stem/progenitor” populations (Reya T et al. Nature, 2005, 434, 843). However, Wnt signaling is also critical in the differentiation processes and development of cells, tissues and organs. These divergent behaviors of Wnt signaling are apparently controlled via selective usage of the coactivator proteins CBP or p300 (Ma et al., Teo et al. and McMillan and Kahn 2005). The endogenous choice of coactivator usage appears to be controlled by a complex array of differential post-translational modifications.

Pharmacologically, the inventor previously showed direct coactivator selection by blocking the CBP/β-catenin interaction with ICG-001, thereby forcing a switch to the p300/catenin interaction, which has divergent promoter specific effects (Ma et al.). The present results demonstrate that IQ-1, through modulation of post-translational modifications and interaction with components of Wnt/β-catenin inhibitory feedback, can also manipulate Wnt/β-catenin coactivator selection. Enhancing Wnt/β-catenin/CBP signaling and preventing the switch to Wnt/β-catenin/p300 usage by IQ-1 allows for the long term expansion of ES cells while maintaining pluripotency in defined media without MEFs or serum. The controlled proliferation of “stem/progenitor” cells for the production of the raw materials required for regenerative medicine is an important outcome of the present invention.

Additional compounds useful for practicing the methods described and claimed herein include compounds disclosed and taught in Japanese patent publication JP2006/180763A2, filed Dec. 27, 2004, which is incorporated by reference herein. Compounds of the patent publication include those designated as compounds with the structures as disclosed in JP2006/180763A2; examples are shown below.

Compounds include the following, designated below as formula (compound) numbers. 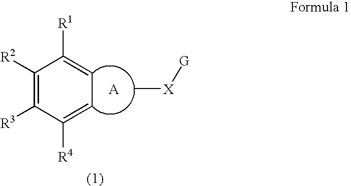
where R¹, R², R³ and R⁴ may be the same or different; and represent an electron attractive group, an electron donating group or a hydrogen atom. Ring A indicates a 5 to 8 member ring containing inside the ring at least one hetero atom. X indicates an alkylene group with 0 to 10 atoms on the main chain. An alkylene group with 0 atoms indicates a single bond. Ethylene configured of at least one the said alkylene groups may be substituted by —C═C— group and/or —N═N— group and/or —CONH— group. In addition, it may be a double bond group which bonds with ring A. In addition, the alkylene group may have at least one electron attractive group, electron donating group or hydrogen atom as a substitution group. G is an aromatic group which may have an electron attractive group, electron donating group or hydrogen group. Said ring A may have at least one electron attractive group and/or electron donating group as a substitution group instead of a —XG group.

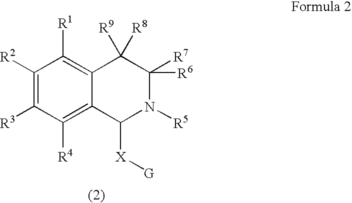

where R¹, R², R³ and R⁴, X and G are the same as defined in Compound 1 above. R⁵, R⁶, R⁷, R⁸ and R⁹ may be the same or they may be different and they indicate an electron attractive group, an electron donating group or a hydrogen atom.

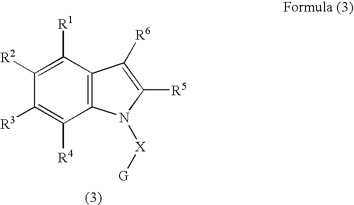

where R¹, R², R³, R⁴, X and G are the same as defined in Compound 1 above. R⁵ and R⁶ may be the same or different and they indicate an electron attractive group, an electron donating group or a hydrogen atom.

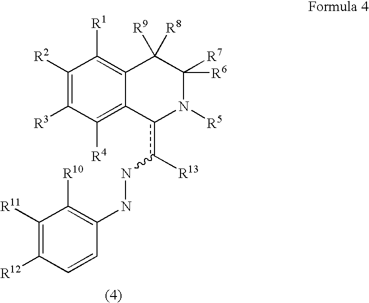

where R¹, R², R³, R⁴, R⁵, R⁶, R⁷, R⁸ and R⁹ are the same as defined above. R¹⁰, R¹¹, R¹² and R¹³ may be the same or different and represent an electron attractive group, an electron donating group or a hydrogen atom. The double single sided broken line indicates a single bond or a double bond. When the double single sided broken line indicates a double bond, a geometrical isomer is present for the wavy line part. There are no particular restrictions on where these geometrical isotropes are disposed and they may be independent of one another, or they may be E bodies or Z bodies.

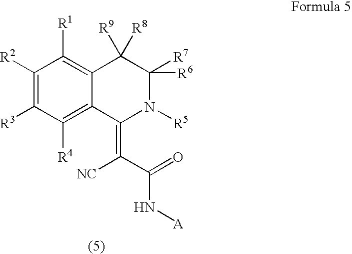

wherein A may be a hydrogen atom or Formula 6:

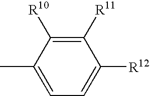

where R¹, R², R³, R⁴, R⁵, R⁶, R⁷, R⁸ and R⁹ are the same as defined above. R¹⁰, R¹¹, R¹² may be the same or different and indicate an electron attractive group, an electron donating group or a hydrogen atom.

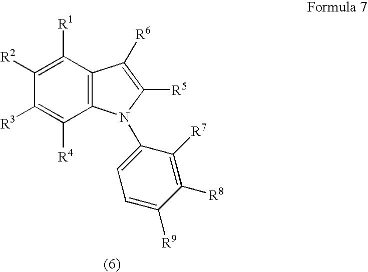

where R¹, R², R³, R⁴, R⁵ and R⁶ are the same as defined above. R⁷, R⁸ and R⁹ may be the same or different and indicate an electron attractive group, an electron donating group or a hydrogen atom.

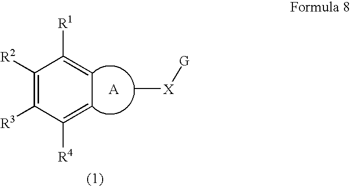

where R¹, R², R³ and R⁴ may be the same or different and represent an electron attractive group, an electron donating group or a hydrogen atom. By “electron donating group” is meant a substitution group which can donate electrons to a benzene ring; by “electron attractive group” is meant a substitution group which has the capacity to attract electron_{π} on a benzene ring. In addition, the electron donating group is defined as _{σ}<o and the electron attractive group is defined as _{σ}>o using Hammett's substitution group constant _{σ}. (Fundamental Organic Reaction Theory, Hashimoto Yasunobu, et al., Sankyo Publishing, 1997).

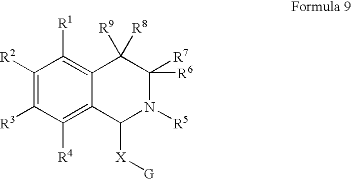

where R¹, R², R³ R⁴ and X and G are defined as above. R⁵, R⁶, R⁷, R⁸ and R⁹ may be the same or different and indicate an electron attractive group, an electron donating group or a hydrogen atom.

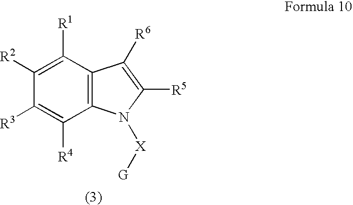

where R¹, R², R³, R⁴ and X and G are defined as above. R⁵ and R⁶ may be the same or different and represent an electron attractive group, an electron donating group or a hydrogen atom.

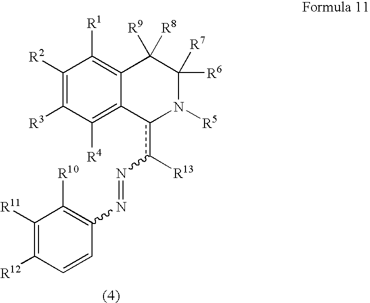

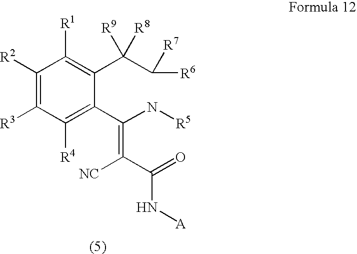

wherein A is a hydrogen atom or Formula 13:

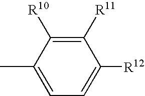

For Formulas 11 and 12, R¹ through R¹³ may be the same or different and they represent an electron attractive group, an electron donating group or a hydrogen atom.

Specific examples include:
2-(4-acetyl-phenyl azo)-2-(3,3-dimethyl-3,4-dihydro-2H-isoquinoline-1-ylidene)-acetamide
2-(3-acetyl-phenyl azo)-2-(3,3-dimethyl-3,4-dihydro-2H-isoquinoline-1-ylidene)-acetamide
2-(4-bromo-phenyl azo)-2-(3,3-dimethyl-3,4-dihydro-2H-isoquinoline-1-ylidene)-acetamide
2-(3-bromo-phenyl azo)-2-(3,3-dimethyl-3,4-dihydro-2H-isoquinoline-1-ylidene)-acetamide
2-(4-chlor-phenyl azo)-2-(3,3-dimethyl-3,4-dihydro-2H-isoquinoline-1-ylidene)-acetamide
2-(3-chlor-phenyl azo)-2-(3,3-dimethyl-3,4-dihydro-2H-isoquinoline-1-ylidene)-acetamide
2-(3,3-dimethyl-3,4-dihydro-2H-isoquinoline-1-ylidene)-2-m-tolyl azo-acetamide
2-(3,3-dimethyl-3,4-dihydro-2H-isoquinoline-1-ylidene)-2-p-tolyl azo-acetamide
2-(3,3-dimethyl-3,4-dihydro-2H-isoquinoline-1-ylidene)-2-(4-methoxy-phenyl azo)-acetamide
2-(3,3-dimethyl-3,4-dihydro-2H-isoquinoline-1-ylidene)-2-(3-methoxy-phenyl azo)-acetamide
2-(3,3-dimethyl-3,4-dihydro-2H-isoquinoline-1-ylidene)-2-(4-nitro-phenyl azo)-acetamide
2-(3,3-dimethyl-3,4-dihydro-2H-isoquinoline-1-ylidene)-2-(3-nitro-phenyl azo)-acetamide
2-(3,3-dimethyl-3,4-dihydro-2H-isoquinoline-1-ylidene)-2-(4-sulfamoyl-phenyl azo)-acetamide
2-(3,3-dimethyl-3,4-dihydro-2H-isoquinoline-1-ylidene)-2-(3,sulfamoyl-phenyl azo)-acetamide
2-(4-acetyl amino-phenyl azo)-2-(3,3-dimethyl-3,4-dihydro-2H-isoquinoline-1-ylidene)-acetamide
2-(3-acetyl amino-phenyl azo)-2-(3,3-dimethyl-3,4-dihydro-2H-isoquinoline-1-ylidene)-acetamide
2-(2-acetyl-phenyl azo)-2-(3,3-dimethyl-3,4-dihydro-2H-isoquinoline-1-ylidene)-acetamide
2-(3,3-dimethyl-3,4-dihydro-2H-isoquinoline-1-ylidene)-2-phenyl azo-acetamide
(4-acetyl-phenyl azo)-(3,3-dimethyl-3,4-dihydro-2H-isoquinoline-1-ylidene)-ethyl acetate ester
2-(4-acetyl-phenyl azo)-2-(3,3-dimethyl-1,2,3,4-tetrahydro-isoquinoline-1-yl)-acetamide
2-(4-acetyl-phenyl azo)-2-(3,3-dimethyl-3,4-dihydro-2H-isoquinoline-1-ylidene)-N-methyl-acetamide
2-(4-acetyl-phenyl azo)-2-(3,3-dimethyl-3,4-dihydro-2H-isoquinoline-1-ylidene)-N-phenyl-acetamide
2-(4-acetyl-phenyl azo)-2-(2,3,3-trimethyl-3,4-dihydro-2H-isoquinoline-1-ylidene)-acetamide
(4-acetyl-phenyl azo)-(3,3-dimethyl-3,4-dihydro-2H-isoquinoline-1-ylidene)-acetonitrile
2-(4-acetyl-phenyl azo)-2-(3,3-dimethyl-3,4-dihydro-2H-isoquinoline-1-ylidene)-N,N-dimethyl-acetamide
(4-acetyl-phenyl azo)-(3,3-dimethyl-3,4-dihydro-2H-isoquinoline-1-ylidene)-acetate
2-(2-acetyl-3,3-dimethyl-3,4-dihydro-2H-isoquinoline-1-ylidene)-2-(4-acetyl-phenyl azo)-acetamide
2-cyano-2-(3,3-dimethyl-3,4-dihydro-2H-isoquinoline-1-ylidene)-N-p-tolyl-acetamide
2-cyano-2-(3,3-dimethyl-3,4-dihydro-2H-isoquinoline-1-ylidene)-N-m-tolyl-acetamide
2-cyano-2-(3,3-dimethyl-3,4-dihydro-2H-isoquinoline-1-ylidene)-N-o-tolyl-acetamide
2-cyano-2-(3,3-dimethyl-3,4-dihydro-2H-isoquinoline-1-ylidene)-N-(4-methoxy-phenyl)-acetamide
2-cyano-2-(3,3-dimethyl-3,4-dihydro-2H-isoquinoline-1-ylidene)-N-(3-methoxy-phenyl)-acetamide
2-cyano-2-(3,3-dimethyl-3,4-dihydro-2H-isoquinoline-1-ylidene)-N-(4-nitro-phenyl)-acetamide
2-cyano-2-(3,3-dimethyl-3,4-dihydro-2H-isoquinoline-1-ylidene)-N-(3-nitro-phenyl)-acetamide
4-[2-cyano-2-(3,3-dimethyl-3,4-dihydro-2H-isoquinoline-1-ylidene-acetyl amino]-ethyl benzoate ester
3-[2-cyano-2-(3,3-dimethyl-3,4-dihydro-2H-isoquinoline-1-ylidene)-acetyl amino]-ethyl benzoate ester
2-cyano-2-(3,3-dimethyl-3,4-dihydro-2H-isoquinoline-1-ylidene)-N-phenyl-acetamide
2-cyano-2-(3,3-dimethyl-3,4-dihydro-2H-isoquinoline-1-ylidene)-N-(2,4-dimethyl-phenyl)-acetamide
2-cyano-2-(3,3-dimethyl-3,4-dihydro-2H-isoquinoline-1-ylidene)-acetamide

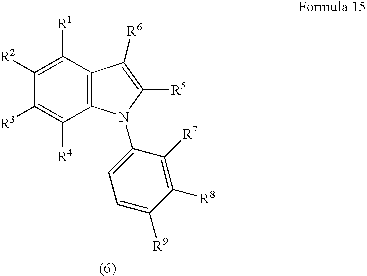

where R¹ through R⁹ are electron attractive group, an electron donating group or a hydrogen atom which may be the same or different. Of these, R¹ through R⁹ should be a group or an atom selected from a group made up of an alkyl group, an alkoxy group, a hydroxyl group, a nitro group, a nitrile group, an acetoxy group, an acetoxy alkyl group, a cyclic alkyl amino alkyl group which may include an oxygen atom, a dialkyl aminoalkyl group, a dialkyl amino vinyl group, a hydroxy alkyl amino alkyl group, an aryl aminovinyl group, an alkoxy carbonyl group, a halogen atom and a hydrogen atom. In addition, R¹ and R² should be a hydrogen atom; R³ should be a hydroxy group or an acetoxy group; R⁴ should be an acetoxy alkyl group, a cyclic alkyl aminoalkyl group which may contain an oxygen atom, a di-lower alkyl amino lower alkyl group, a hydroxy lower alkyl amino lower alkyl group or a hydrogen atom; R⁵ should be a lower alkyl group, a di-lower alkyl amino vinyl group or an aryl amino vinyl group; R⁶ should be a nitro group; R⁷, R⁸ and R⁹ should be a lower alkyl group, a lower alkoxy group or a hydrogen atom which may be the same or different.

Specific examples of the compound include:
2-methyl-3-nitro-1-phenyl-1H-indole-6-ol
1-(4-methoxy-phenyl)-2-methyl-3-nitro-1H-indole-6-ol
2-methyl-3-nitro-1-p-tolyl-1H-indole-6-ol
2-[2-(4-methoxy-phenyl amino)-vinyl]-3-nitro-1-p-tolyl-1H-indole-6-ol
1-(2-methoxy-phenyl)-2-methyl-3-nitro-1H-indole-6-ol
7-dimethyl amino methyl-2-(2-dimethyl amino-vinyl)-3-nitro-1-p-tolyl-1H-indole-6-ol
1-(4-methoxy-phenyl)-2-methyl-3-nitro-7-piperidine-1-yl methyl-1H-indole-6-ol-hydrochloride
2-(2-dimethyl amino-vinyl)-1-(4-methoxy-phenyl)-7-morpholine-4-yl methyl-3-nitro-1H-indole-6-ol
7-[(3-hydroxy-propyl amino)-methyl]-1-(4-methoxy-phenyl)-2-methyl-3-nitro-1H-indole-6-ol hydrochloride
7-dimethyl amino methyl-2-(2-dimethyl amino vinyl)-1-(4-methoxy-phenyl)-3-nitro-1H-indole-6-ol
7-diethyl amino methyl-1-(4-methoxy-phenyl)-2-methyl-3-nitro-1H-indole-6-ol
7-dimethyl amino methyl-2-methyl-3-nitro-1-p-tolyl-1H-indole-6-ol
1-(4-methoxy-phenyl)-2-methyl-3-nitro-7-piperidine-1-yl methyl-1H-indole-6-ol acetate
7-acetoxy methyl-2-methyl-3-nitro-1-p-tolyl-1H-indole-6-yl ester
2-(2-dimethyl amino vinyl)-1-(4-methoxy-phenyl)-3-nitro-7-piperidine-1-yl methyl-1H-indole-6-ol
7-dimethyl amino methyl-2-methyl-3-nitro-1-phenyl-1H-indole-6-ol
7-dimethyl aminomethyl-1-(4-methoxy-phenyl)-2-methyl-3-nitro-1H-indole-6-ol acetate
6-acetoxy-1-(4-methoxy-phenyl)-2-methyl-3-nitro-1H-indole-7-yl methyl ester
2-(2-dimethyl amino-vinyl)-3-nitro-1-p-tolyl-1H-indole-6-ol
2-(2-dimethyl amino-vinyl)-3-nitro-1-phenyl-1H-indole-6-ol
acetate 6-acetoxy-2-(2-dimethyl amino-vinyl)-1-(4-methoxy-phenyl)-3-nitro-1H-indole-7-yl methyl ester
1-(4-chlor-phenyl)-2-methyl-3-nitro-1H-indole-6-ol
acetate 2-(2-dimethyl amino-vinyl)-6-hydroxy-1-(4-methoxyl-phenyl)-3-nitro-1H-indole-7-il methyl ester
5-hydroxy-2-methyl-4,6-dinitro-1-phenyl-1H-indole-3-ethyl carbonate ester
7-[[bis-(2-hydroxy-ethyl)-amino]-methyl]-1-(4-methoxy-phenyl)-2-methyl-3-nitro-1H-indole-6-ol
7-[[bis-(2-hydroxy-ethyl)-amino]-methyl]-2-methyl-3-nitro-1-p-tolyl-1H-indole-6-ol
7-dimethyl amino methyl-2-(2-dimethyl amino-vinyl)-3-nitro-1-phenyl-1H-indole-6-ol
2-(6-hydroxy-3-nitro-1-phenyl-1H-indole-2-yl methyl)-isothio urea
acetate 2-(N,N′-diphenyl-carbamimide yl sulfanyl methyl)-1-(4-methoxy-phenyl)-3-nitro-1H-indole-6-yl ester
acetate 6-acetoxy-1-(4-acetoxy-phenyl)-2-(dimethyl amino-vinyl)-3-nitro-1H-indole-7-yl methyl ester
2-(2-dimethyl amino-5-hydroxy-benzofurane-3-yl)-1-(4-methoxy-phenyl)-3-nitro-1H-indole-6-ol
2-(2-dimethyl amino-vinyl)-1-(4-methoxy-phenyl)-3-nitro-1H-indole-6-ol
5-bromo-1-(4-methoxy-phenyl)-2-methyl-3-nitro-1H-indole-6-ol
acetate 1-(4-methoxy-phenyl)-2-methyl-3-nitro-1H-indole-6-ol
7-dimethyl amino methyl-6-hydroxy-2-methyl-1-phenyl-1H-indole-3-carbonitrile
7-diethyl amino methyl-6-hydroxy-2-methyl-1-phenyl-1H-indole-3-carbonitrile
2-(2-dimethyl amino-5-hydroxy-benzofurane-3-yl)-6-methoxy-1-phenyl-1H-indole-3-carbonitrile
6-methoxy-2-methyl-1-phenyl-1 H-indole-3-carbonitrile 2-(2-dimethyl amino-vinyl)-6-methoxy-1-phenyl-1H-indole-3-carbonitrile
5-bromo-6-hydroxy-1-(4-methoxy-phenyl)-2-methyl-1H-indole-3-carbonitrile
5,7-dibromo-6-hydroxy-1-(4-methoxy-phenyl)-2-methyl-1H-indole-3-carbonitrile
6-hydroxy-1-(4-methoxy-phenyl)-2-methyl-1H-indole-3-carbonitrile
6-hydroxy-2-methyl-1-p-tolyl-1H-indole-3-carbonitrile
6-hydroxy-2-methyl-1-phenyl-1H-indole-3-carbonitrile
1H-furo[2,3-g]indole-3-acetate, 5-hydroxy-1-(4-methoxy phenyl)-2,8-dimethyl, ethyl ester
5-bromo-7-dimethyl aminomethyl-6-hydroxy-1-phenyl-2-phenyl sulfanyl methyl-1 H-indole-3-ethyl acetate ester
6-hydroxy-2-methyl-1-phenyl-1H-indole-3-acetate
5-bromo-6-hydroxy-1-phenyl-2-phenyl sulfanyl methyl-1H-indole-3-ethyl acetate ester
6-acetoxy-5-bromo-2-methyl-1-phenyl-1H-indole-3-ethyl acetate ester
5-bromo-7-dimethyl amino methyl-6-hydroxy-1-phenyl-2-phenyl sulfanyl methyl-1H-indole-3-ethyl acetate ester.
6-acetoxy-5-bromo-2-bromomethyl-1-phenyl-1H-indole-3-ethyl acetate ester
6-acetoxy-2-bromo methyl-1-phenyl-1H-indole-3-ethyl acetate ester
6-acetoxy-2-methyl-1-phenyl-1H-indole-3-ethyl acetate ester
pyrrolo [2,3-f][1,3]benzoxadine-3-acetate, 8-ethyl-1,7,8,9-tetrahydro-2-methyl-1-(4-nitrophenyl)-, ethyl ester
6-acetoxy-2-methyl-1-(2-trifluoromethyl-phenyl)-1H-indole-3-ethyl acetate ester
1-(2,4-dimethoxy-phenyl)-6-hydroxy-2-methyl-1H-indole-3-ethyl acetate ester
6-hydroxy-1-(4-methoxy-phenyl)-2-methyl-1H-indole-3-ethyl acetate ester
1-(4-ethoxy carbonyl-phenyl)-6-hydroxy-2-methyl-1H-indole-3-ethyl acetate ester
1-(4-cyano-phenyl)-6-hydroxy-2-methyl-1H-indole-3-ethyl acetate ester
6-hydroxy-2-methyl-1-(2-trifluoro methyl-phenyl)-1H-indole-3-ethyl acetate ester
6-hydroxy-2-methyl-1-(4-trifluoro methyl-phenyl)-1H-indole-3-ethyl acetate ester
6-hydroxy-2-methyl-1-(4-nitro-phenyl)-1H-indole-3-ethyl acetate ester
1-(4-bromo-phenyl)-6-hydroxy-2-methyl-1H-indole-3-ethyl acetate ester
1-(4-fluoro-phenyl)-6-hydroxy-2-methyl-1H-indole-3-ethyl acetate ester
6-hydroxy-2-methyl-1-(4-nitro-phenyl)-5,7-bis-piperidine-1-yl methyl-1H-indole-3-ethyl acetate ester
5,7-bis-dimethyl amino methyl-6-hydroxy-2-methyl-1-(4-nitro-phenyl)-1H-indole-3-ethyl acetate ester
6-hydroxy-2-methyl-1-(4-nitro-phenyl)-1H-indole-3-ethyl acetate ester
6-acetoxy-1-(4-chlor-phenyl)-2-methyl-1H-indole-3-ethyl acetate ester
7-dimethyl amino methyl-6-hydroxy-2-methyl-1-(4-nitro-phenyl)-1H-indole-3-ethyl acetate ester
7-dimethyl amino methyl-6-hydroxy-2-methyl-1-phenyl-1H-indole-3-ethyl acetate ester
6-hydroxy-2-methyl-1-p-tolyl-1H-indole-3-ethyl acetate ester
6-hydroxy-2-methyl-1-phenyl-1H-indole-3-ethyl acetate ester
1-(4-chlor-phenyl)-7-dimethyl amino methyl-6-hydroxy-2-methyl-1H-indole-3-ethyl acetate ester
1-(4-dimethyl amino-phenyl)-6-hydroxy-2-methyl-1H-indole-3-ethyl acetate ester
1-(2-chlor-phenyl)-6-methoxy-2-methyl-1H-indole-3-ethyl acetate ester
1-(2-chlor-phenyl)-6-hydroxy-2-methyl-1H-indole-3-ethyl acetate ester
6-acetoxy-1-(4-chlor-phenyl)-2-methyl-5,7-dinitro-1H-indole-3-ethyl acetate ester
1-(4-chlor-phenyl)-6-hydroxy-2-methoxy-2-methyl-5,7-dinitro-1H-indole-3-ethyl acetate ester
6-acetoxy-5,7-dibromo-1-(4-chlor-phenyl)-2-methyl-1H-indole-3-ethyl acetate ester
5,7-dibromo-1-(4-chlor-phenyl)-6-hydroxy-2-methyl-1H-indole-3-ethyl acetate ester
6-acetoxy-5-bromo-1-(4-chlor-phenyl)-2-methyl-1H-indole-3-ethyl acetate ester
5-bromo-1-(4-chlor-phenyl)-6-methoxy-2-methyl-1H-indole-3-ethyl acetate ester
1-(4-chlor-phenyl)-6-methoxy-2-methyl-1H-indole-3-acetate
1-(4-chlor-phenyl)-6-methoxy-2-methyl-1H-indole-3-ethyl acetate ester
1-(4-chlor-phenyl)-6-hydroxy-2-methyl-1H-indole-3-ethyl acetate ester

Examples of the lower alkyl group represented by R are as follows: methyl, ethyl, n-propyl, isopropyl, n-butyl, isobutyl, sec-butyl, tert-butyl, n-pentyl, isopentyl, neopentyl, 1-methyl propyl, n-hexyl, isohexyl, 1,1-dimethyl butyl, 2,2-dimethyl butyl, 3,3-dimethyl butyl, 3,3-dimethyl propyl, 2-ethyl propyl and the like. Methyl is especially suitable. Examples of the lower alkoxy group represented by R are as follows: methoxy, ethoxy, propoxy, isopropoxy, butoxy, sec-butoxy, tert-butoxy, pentoxy, hexyloxy, heptyloxy, octyloxy and the like. Methoxy is especially suitable.

Examples of the halogen atom represented by R are as follows: fluorine, chlorine, bromine, iodine and the like but chlorine or bromine are especially suitable. Examples of the lower acyl group represented by R are as follows: formyl, acetyl, propionyl, butyryl and the like and acetyl is especially suitable. Examples of the lower alkyl group which may form the cyclic structure represented by R are as follows: cyclobutyl, cyclopentyl, cyclohexyl, cycloheptyl, cyclooctyl and the like. Cyclopentyl, cyclohexyl and cycloheptyl are especially suitable.

Examples of the lower alkoxy carbonyl represented by R are as follows: methoxy carbonyl, ethoxy carbonyl, propoxy carbonyl and the like, with methoxy carbonyl or ethoxy carbonyl being especially suitable. Examples of the amino carbonyl represented by R are as follows: —CONR₂ (R is a hydrogen atom which may be the same or different; it represents the lower alkyl group illustrated previously, and a phenyl group which may have a substitution group).

Examples of the secondary aminocarbonyl group represented by R are —CONHR (indicates a lower alkyl group illustrated previously and a phenyl group which may have a substitution group). In addition, the tertiary amino carbonyl group may be —CONR₂ (R represents the lower alkyl group which was illustrated previously which may be the same or different, and a phenyl group which may have a substitution group). Examples of the amino alkyl group represented by R are: —(CH₂)ₙ—NR₂ (where n is an integer from 1 to 8 and preferably 1). R is a hydrogen atom, a lower alkyl group, or a lower alkyl group which may form a cyclic structure which may be the same or different (1 to 3 hetero atoms may be included in the nitrogen and oxygen in the cyclic structure), and a phenyl group which may have a substitution group) and the like.

An example of the acetoxy alkyl represented by R is: —(CH₂)ₙ-Oac (where n is an integer from 1 to 8) and n is preferably 1.

Other specific examples include Compounds of formulas 17-28 below.

### 3,3-dimethyl-1,2,3,4-tetrahydroisoquinolinidene-1-acetamide (formula 17)

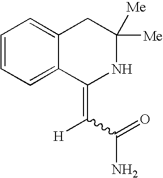

### 2-(4-acetyl-phenyl azo)-2-(3,3-dimethyl-3,4-dihydro-2H-isoquinoline-1-ylidene)-acetamide (formula 18)

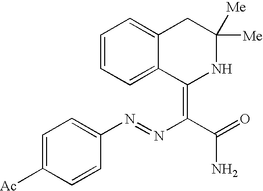

### 2-(3-acetyl-phenyl azo)-2-(3,3-dimethyl-3,4-dihydro-2H-isoquinoline-1-ylidene)-acetamide (formula 19)

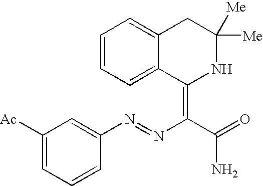

### 2-(4-acetyl-phenyl azo)-2-(2,3,3-trimethyl-3,4-dihydro-2H-isoquinoline-1-ylidene)-acetamide (formula 20)

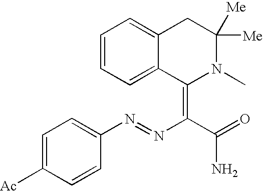

### 2-(2-acetyl-3,3-dimethyl-3,4-dihydro-2H-isoquinoline-1-ylidene)-2-(4-acetyl-phenyl azo)-acetamide (formula 21)

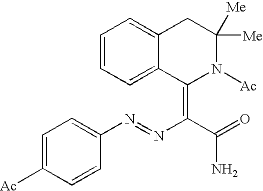

### (4-acetyl-phenyl azo)-(3,3-dimethyl-3,4-dihydro-2H-isoquinoline-1-ylidene)-acetonitrile (formula 22)

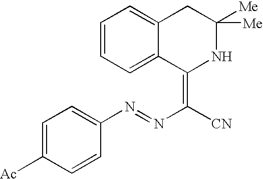

### (4-acetyl-phenyl azo)-(3,3-dimethyl-3,4-dihydro-2H-isoquinoline-1-ylidene)-acetate (formula 23)

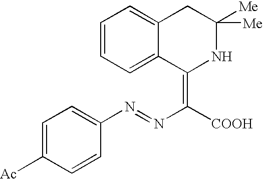

### (4-acetyl-phenyl azo)-(3,3-dimethyl-3,4-dihydro-2H-isoquinoline-1-ylidene)-ethyl acetate ester (formula 24)

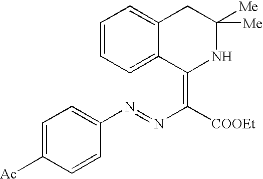

### 2-(4-acetyl-phenyl azo)-2-(3,3-dimethyl-3, 4-dihydro-2H-isoquinoline-1-ylidene)-N-methyl-acetamide (formula 25)

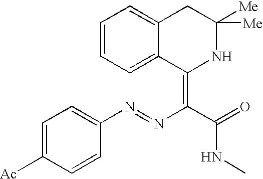

### 2-(4-acetyl-phenyl azo)-2-(3,3-dimethyl-3,4-dihydro-2H-isoquinoline-1-ylidene)-N,N-dimethyl-acetamide (formula 26)

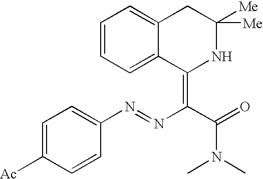

### 2-(4-acetyl-phenyl azo)-2-(3,3-dimethyl-3,4-dihydro-2H-isoquinoline-1-ylidene)-N-phenyl-acetamide (formula 27)

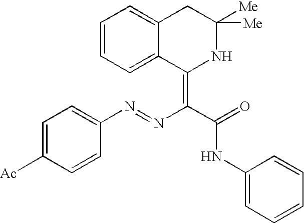

### 2-(4-acetyl-phenyl azo)-2-(3,3-dimethyl-1,2,3,4-tetrahydro-isoquinoline-1-yl)-acetamide (formula 28)

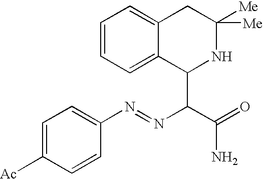

Other examples of compounds include:
2-cyano-2-(3,3-dimethyl-3,4-dihydro-2H-isoquinoline-1-ylidene)-N-p-tolyl-acetamide
2-(4-acetyl-phenyl azo)-2-(3,3-dimethyl-3,4-dihydro-2H-isoquinoline-1-ylidene)-acetamide
2-(3-chlor-phenyl azo)-2-(3,3-dimethyl-3,4-dihydro-2H-isoquinoline-1-ylidene)-acetamide
2-(3,3-dimethyl-3,4-dihydro-2H-isoquinoline-1-ylidene)-2-(3-methoxy-phenyl azo)-acetamide
2-(3,3-dimethyl-3,4-dihydro-2H-isoquinoline-1-ylidene)-2-(3-nitro-phenyl azo)-acetamide
2-(3,3-dimethyl-3,4-dihydro-2H-isoquinoline-1-ylidene)-2-(4-tolyl-azo-phenyl azo)-acetamide
2-(3-acetyl-phenyl azo)-2-(3,3-dimethyl-3,4-dihydro-2H-isoquinoline-1-ylidene)-acetamide
2-(3,3-dimethyl-3,4-dihydro-2H-isoquinoline-1-ylidene)-2-m-tolyl-azo-acetamide 2-methyl-3-nitro-1-phenyl-1H-indole-6-ol
1-(4-methoxy-phenyl)-2-methyl-3-nitro-1H-indole-6-ol 2-methyl-3-nitro-1-p-tolyl-1H-indole-6-ol
2-[2-(4-methoxy-phenyl amino)-vinyl]-3-nitro-1-p-tolyl-1H-indole-6-ol 1-(2-methoxy-phenyl)-2-methyl-3-nitro-1H-indole-6-ol
7-dimethyl amino methyl-2-(2-dimethyl amino-vinyl)-3-nitro-1-p-tolyl-1H-indole-6-ol
1-(4-methoxy-phenyl)-2-methyl-3-nitro-7-piperidine-1-yl methyl-1H-indole-6-ol
2-(2-dimethyl amino-vinyl)-1-(4-methoxy-phenyl)-7-morpholine-4-yl methyl-3-nitro-1H-indole-6-ol
7-[(3-hydroxy-propyl amino)-methyl]-1-(4-methoxy-phenyl)-2-methyl-3-nitro-1H-indole-6-ol
7-dimethyl amino methyl-2-(2-dimethyl amino-vinyl)-1-(4-methoxy-phenyl)-3-nitro-1H-indole-6-ol
7-dimethyl amino methyl-1-(4-methoxy-phenyl)-2-methyl-3-nitro-1H-indole-6-ol
7-dimethyl amino methyl-2-methyl-3-nitro-1-p-tolyl-1H-indole-6-ol
1-(4-methoxy-phenyl)-2-methyl-3-nitro-7-piperidine-1-yl methyl-1H-indole-6-ol
acetate 7-acetoxy methyl-2-methyl-3-nitro-1-p-tolyl-1H-indole-6-ol ester
2-(2-dimethyl amino-vinyl)-1-(4-methoxy-phenyl)-3-nitro-7-piperidine-1-yl methyl-1H-indole-6-ol
7-dimethyl amino methyl-2-methyl-3-nitro-1-phenyl-1H-indole-6-ol
7-dimethyl amino methyl-1-(4-methoxy-phenyl)-2-methyl-3-nitro-1H-indole-6-ol
acetate 6-acetoxy-1-(4-methoxy-phenyl)-2-methyl-3-nitro-1H-indole-7-yl methyl ester
2-(2-dimethyl amino-vinyl)-3-nitro-1-p-tolyl-1H-indole-6-ol
2-(2-dimethyl amino-vinyl)-3-nitro-1-phenyl-1H-indole-6-ol
acetate 6-acetoxy-2-(2-dimethyl amino-vinyl)-1-(4-methoxy-phenyl)-3-nitro-1H-indole-7-yl methyl ester
1-(4-chlor-phenyl)-2-methyl-3-nitro-1H-indole-6-ol
acetate 2-(2-dimethyl amino-vinyl)-6-hydroxy-1-(4-methoxy-phenyl)-3-nitro-1H-indole-7-yl methyl ester
5-hydroxy-2-methyl-4,6-dinitro-1-phenyl-1H-indole-3-ethyl carboxylate ester

Such compounds can be tested as described in the Examples herein for their suitability for practicing the methods claimed and disclosed herein.

It should be understood that the examples and embodiments described herein are for illustrative purposes only and that various modifications or changes in light thereof will be suggested to persons skilled in the art and are to be included within the spirit and purview of this application.

### EXAMPLES

### Experimental Procedures used in Examples 1-7

Cell culture. ESCs, D3ES (ATCC CRL-1934) were maintained on Mitomycin C treated MEFs in mouse ESC medium containing DMEM (Invitrogen) supplemented with 15% FBS (Invitrogen), 0.1 mM MEM nonessential amino acids (Invitrogen), 0.1 mM 2-mercaptoethanol (Sigma), 2 mM L-glutamine (Invitrogen) and 1,000 U/ml LIF (CHEMICON). To remove MEFs, cells were collected by trypsinization and plated on gelatin-coated culture dishes for 20 min. Non-adherent cells consisting mainly of ESCs were replated on gelatin-coated culture dishes again. Non-adherent cells were used for further experiments. P19 cells were cultured according to conditions recommended by the ATCC. Cells were incubated at 37° C. in a 5% CO₂ incubator. Purified Wnt3a was purchased (R&D Systems).

Compound Screens. For the small molecule screen, alkaline phosphatase activity of ESCs was determined. ESCs were plated into 96-well tissue culture plates (Falcon) at a density of 316-1,000 cells per well in ESC medium without LIF. Compounds were added at a final concentration of 4 μg/ml. Cells were treated with compounds for 7 days and test cell populations were washed with PBS and 100 μl of p-nitrophenyl phosphate solution (MOSS Inc.) was added. The absorbance at 405 nm was measured by spectrophotometer (Spectramax, Molecular Devices).

Flow Cytometry. Analysis of SSEA-1 expression of ESCs was performed by flow cytometry according to Zandstra et al. (35). The test cell population was washed in ice cold Hanks balanced salt solution (Invitrogen) containing 2% FCS(HF) and resuspended for 10 min in HF containing anti-mouse CD16/CD32 monoclonal antibody at 1 μg/100 μl (Pharmingen) to block nonspecific binding. Blocked cells were then incubated at 1×10⁷ cells/ml for 40 min on ice with anti-SSEA-1 (Kyowa Medex) followed by FITC-Goat anti Mouse IgM antibody (ZYMED). Cells to be analyzed for their SSEA-1 expression were then washed twice in HF with 2 μg/ml propidium iodide (Dojindo) added into the final wash. The cells were then resuspended in HF for analysis on a FACS Calibur (Becton Dickinson).

NIH-3T3 cells (wt), NIH-3T3 CBP(+/−) cells and NIH-3T3 p300(+/−) cells were transfected as described in (26). Briefly, cells were transfected with TOPFLASH or FOPFLASH, using Fugene6 (Roche Molecular Biochemicals). Transfection efficiencies were normalized with pRL-null luciferase plasmid. Luciferase assays were performed with the DUAL-Luciferase Reporter Assay System (Promega).

Real-time RT-PCR. Total RNA was isolated and reverse-transcribed using SuperScript III (Invitrogen). Real-time RT-PCR (Sybr Green; BioRad) was performed using with the gene-specific primers:

| | | |
|---|---|---|
| Nanog-F | agggtctgctactgagatgctctg | (SEQ ID NO:5) |
| Nanog-R | caaccactggtttttctgccaccg | (SEQ ID NO:6) |
| GAPDH-F | ggtgaaggtcggtgtgaacgga | (SEQ ID NO:7) |
| GAPDH-R | tgttagtggggtctcgctcctg | (SEQ ID NO:8) |

In certain experiments, 2 μg of extracted total RNA was reverse-transcribed using SuperScript III RT-PCR system (Invitrogen) according to the manufacturer's protocol. 1 μl of cDNA was amplified by PCR with one or more of the gene-specific primers of SEQ ID NOs:5-18 using optimized PCR cycles to obtain amplified reactions in the linear range.

| | | |
|---|---|---|
| Oct-3/4-F | ggcgttctctttggaaaggtgttc | (SEQ ID NO:9) |
| Oct-3/4-R | ctcgaaccacatccttctct | (SEQ ID NO:10) |
| Rex-1-F | gtcttatcgatgctggagtg | (SEQ ID NO:11) |
| Rex-1-R | aaagctcttctcgcagccat | (SEQ ID NO:12) |
| Sox-2-F | gcatgtcctactcgcagcag | (SEQ ID NO:13) |
| Sox-2-R | gctgatcatgtcccggaggt | (SEQ ID NO:14) |
| C-Myc-F | accaacagcaactatgacctc | (SEQ ID NO:15) |
| C-Myc-R | aaggacgtagcgaccgcaac | (SEQ ID NO:16) |
| MDR-1-F | tgcttatggatcccagagtga | (SEQ ID NO:17) |
| MDR-1-R | ttggtgaggatctctccgcgt | (SEQ ID NO:18) |

Transfection and Luciferase assay. ESCs were cultured in 96-well cell culture dishes coated with a 0.1% aqueous gelatin solution and transfected with 0.2 μg/well of pSTAT3-TA-Luc (CLONTECH), using Lipofectamine 2000 (Invitrogen). After 6 hours, cells were washed and exposed to either IQ-1 at the indicated doses, or LIF, for 24 hours. Transfection efficiencies were normalized with pRL-null luciferase plasmid. Luciferase assays were performed with the DUAL-Luciferase Reporter Assay System (Promega).

In certain experiments, NIH-3T3 cells (wt), NIH-3T3 CBP(+/−) cells and NIH-3T3 p300(+/−) cells were transfected as described in (26). Briefly, cells were transfected with TOPFLASH or FOPFLASH, using Fugene6 (Roche Molecular Biochemicals). Transfection efficiencies were normalized with pRL-null luciferase plasmid. Luciferase assays were performed with the DUAL-Luciferase Reporter Assay System (Promega).

Detection of CBP, p300, and phospho-serine89-p300 in P19 cells. P19 cells exposed to either Wnt3A or vehicle control, after which IQ-1 was added to a final concentration of 10 μM. Control DMSO was 0.025%. Cells were incubated for 24 hours. At the end of this incubation period, cells were washed, lysed and subjected to SDS-PAGE. CBP and p300 were detected using the rabbit polyclonal antibody (A-22)(1:5000); and (C-20)(1:5000) (Santa Cruz), respectively. Phospho-serine89-p300 was detected using custom antisera at a dilution of 1:100.

Co-immunoprecipitation of β-catenin with CBP or p300 in P19 cells. Co-immunoprecipitation was performed as described in (27). Briefly, P19 cells were treated with 10 μM ICG-001 or 10 μM IQ-1 (control DMSO was 0.025%) for 24 hours, after which they were washed and lysed using. Nuclear fraction was isolated and precleared with antibodies to CBP(A-22) or p300(N-15). β-catenin was detected using a mouse monoclonal antibody (Becton Dickinson) at a dilution of 1:5000.

Affinity of in vitro phosphorylated p300 with β-catenin. Recombinant p300 (1-110 aa) fused to an HA-tag were incubated with PKCα in kinase buffer (20 mM Hepes, pH 7.4, 10 mM magnesium acetate, 1 mM dithiothreitol, 100 μM ATP). Co-immunoprecipitation was carried out in P19 lysates mixed with the in vitro phosphorylated p300 using an HA-tag antibody. p300 was detected using the rabbit polyclonal antibody (C20), 1:5000. β-catenin was detected using a mouse monoclonal antibody (Becton Dickinson) at a dilution of 1:5000

Affinity Purification of Target Proteins. P19 cells were cultured to 90-100% confluency. Cells were lysed in protein-binding buffer [PBB, 20 mM Hepes, pH 7.9/100 mM NaCL/0.5 mM EDTA/0.5% Nonidet P-40/6 mM MgCl2/5 mM 2-mercaptoethanol/One tablet of Complete protease inhibitor mixture (Roche Molecular Biochemicals)]. Biotinylated IQ-1 was bound overnight at room temperature to a 50% slurry of streptavidin-agarose beads (Amersham Pharmacia) in buffer containing 50% DMSO and 50% PBB. Beads were washed to remove unbound IQ-1 and then incubated with whole cell lysates. Proteins eluted by boiling in SDS were Coomassie stained to detect target proteins, or immunoblotted.

Zebrafish experiments. Wild-type (AB) zebrafish strain at 1-cell stage were treated with IQ-1 at a final concentration of 1 μM in embryo medium for 24 h. Control zebrafish embryos were incubated at an equivalent concentration of DMSO. At the end of this 24 h treatment, embryos were manually dechorionated and imaged. Zebrafish embryos were maintained at 28° C. Results shown are representative of those obtained from at least 10 embryos for each group, from 2 independent experiments.

Immunocytochemistry and Antibodies. Cells were fixed for 20 min with 4% paraformaldehyde in PBS. Immunostaining was carried out using standard protocols. Primary antibodies were used at the following dilutions: anti α-fetoprotein mouse monoclonal (R&D systems, 10 ug/ml), anti-Actin, Smooth muscle Ab-1 mouse monoclonal (LAB VISION, 1:1), anti-GATA4 mouse monoclonal (SANTA CRUZ, 1:100), anti-MAP (microtubule associated protein)₂ mouse monoclonal (Chemicon, 1:200), βIII-tubulin mouse monoclonal (Chemicon, 1:200), anti-Oligodendrocyte mouse monoclonal (Chemicon, 1:1,000). Secondary antibodies were Alexa Fluor 488 or 594 goat anti-mouse IgG (H+L)(Invitrogen, 1:200). Cells were imaged using an Olympus IX 70 microscope with ×40-200 magnification.

### Example 1

### PR72/130 and ES Cell Proliferation

The small molecule IQ-1 (Asahi Kasei Pharma, FIG. 1) selectively increased β-catenin's usage of CBP as a coactivator at the expense of the use of p300, thereby maintaining the embryonic (ES) cells in an undifferentiated state. The combination of Wnt3a and IQ-1 allowed for proliferation and maintenance of pluripotency as judged by the expression of Oct4, Nanog and Rex1, whereas IQ-1 alone was not sufficient to cause proliferation and maintain pluripotency of ES cells.

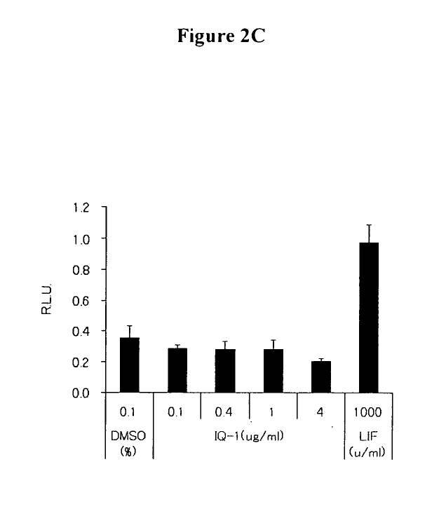

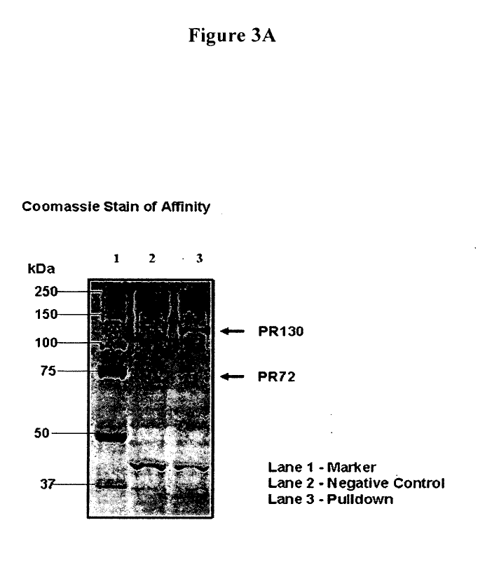

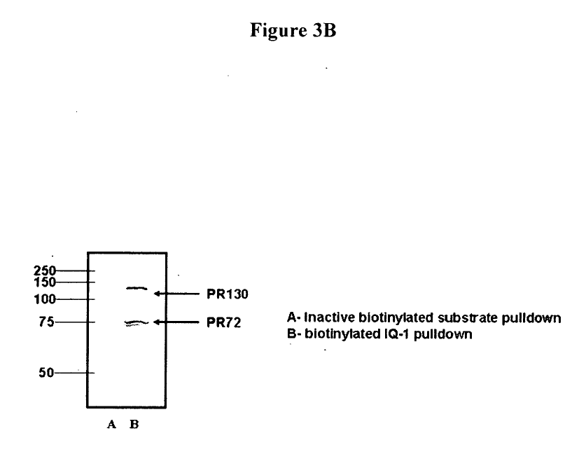

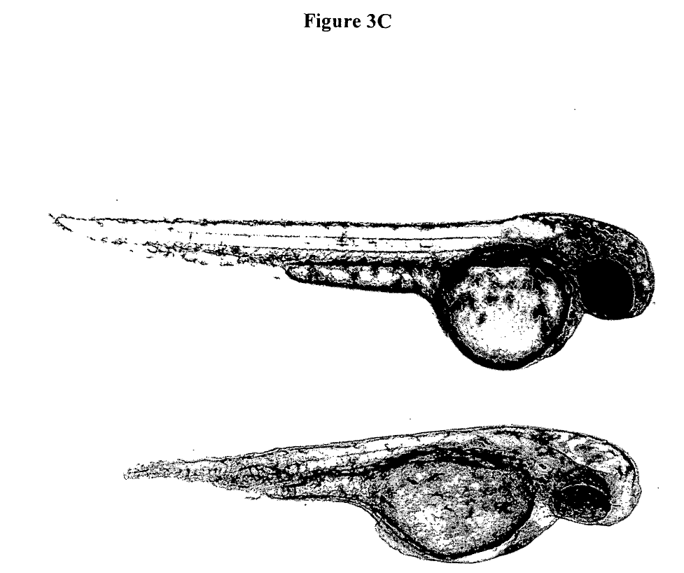

The present example relates to the identification of the molecular target of IQ-1. To identify the molecular target of IQ-1, whole cell lysates from P19 embryonic carcinoma cells were treated with biotinylated IQ-1. Compared to a control biotinylated compound (FIG. 3A, lane 2), biotinylated IQ-1 selectively bound three proteins (FIG. 3A, lane 3). The two bands at 72 and 130 kDa were identified by mass spectral sequencing as the differentially spliced regulatory subunits PR72/130 of the serine/threonine protein phosphatase, PP2A. This was subsequently confirmed by immunoblotting (FIG. 3B).

The fact that IQ-1 selectively binds to these proteins correlates well with the effects seen on the modulation of Wnt signaling, as it has previously been shown that PR72/130 interacts with the protein Naked cuticle, a component of the Wnt signaling pathway, thereby regulating Wnt signaling (Creyghton, M. P. et al., Genes and Dev 19:376-386 2005). Using morpholinos in xenopus embryos, it was also demonstrated that PR72, like Naked cuticle (Nkd), is a negative regulator of Wnt/β-catenin signaling and involved in the switch to non-canonical convergent extension (Creyghton et al.).

### Example 2

### IQ-1 Maintained the Undifferentiated State of ESCs

Murine ESCs (D3 ES) were screened with a chemical library (Asahi Kasei) to identify compounds that enhanced Alkaline Phosphatase (AP) production, a marker of undifferentiated ESCs (12). From this screen, IQ-1 (MW=362.42, FIG. 1A) was identified, which dose dependently increased AP activity (FIG. 1B), in media containing 15% FCS without the addition of exogenous leukemia inhibitory factor (LIF). Treatment with IQ-1 resulted in enhanced expression of the undifferentiated ESC marker, Stage Specific Embryonic Antigen 1 (SSEA-1) in a dose dependent fashion (FIG. 1C).

IQ-1 allowed for long term expansion of ESCs in culture without MEFs and without the addition of exogenous LIF. Mouse D3 ESCs were cultured in media containing 15% FCS plus 4 μg/ml IQ-1 for 65 days. The ESCs continued to proliferate at a steady rate with an approximate 2 log increase every ten days (FIG. 1D).

### Example 3

### IQ-1 Maintained Murine ESC Self-Renewal Independently of LIF

The cytokine LIF, by activating the Stat3 signal transduction pathway, maintains murine ESC symmetrical self-renewal and blocks differentiation (13, 14, 15). LIF did not maintain human ESC self-renewal (16, 17). Nanog is a divergent homeoprotein pluripotency sustaining factor for ESCs (18, 19). Nanog has been shown to act in parallel with LIF-driven stimulation of Stat3 to drive ESC self-renewal. Additionally, elevated expression of Nanog is sufficient for clonal expansion of ESCs and maintenance of expression of the key stem cell transcription factor Oct4, in the absence of Stat3 activation (19).

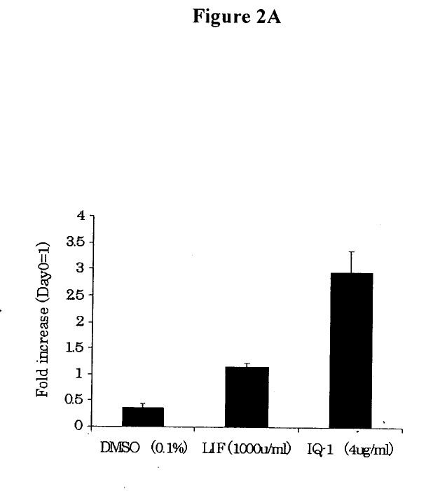

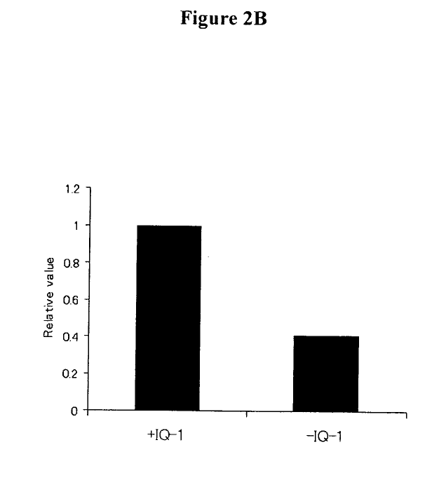

To further investigate the mechanism of action of IQ-1, the effects of IQ-1 on Nanog expression were determined. Whereas feeder-free ESCs treated with LIF only slightly increased Nanog levels, IQ-1 significantly increased and maintained Nanog expression in culture as judged by real time RT-PCR (FIG. 2A). Removal of IQ-1 from culture led to a precipitous drop in Nanog level (FIG. 2B). Loss of Nanog expression has been previously correlated with ESC differentiation (18, 19). To further confirm that the effects of IQ-1 were not mediated via Stat3 signaling, a Stat3/luciferase reporter gene construct was utilized. While IQ-1 significantly elevated Nanog expression, it did not affect Stat3/luciferase expression unlike LIF, which as anticipated, elicited a significant response (FIG. 2C). From this Example, it can be concluded that the affects of IQ-1 on the maintenance of murine ESC pluripotency are independent of the LIF/Stat3 pathway.

### Example 4

### IQ-1 Modulates WNT Signaling Via Interaction with PR72/130

This example was performed to examine the effects of IQ-1 on “non-canonical” Wnt signaling by looking at convergent extension during zebrafish (danio rerio) development. Zebrafish embryos treated with 1 μM IQ-1 showed significant developmental defects (FIG. 3C). In particular a shortened tail, consistent with inhibition of convergent extension and similar to the effects of PR72 morpholinos, was observed (25). From this Example and Example 1, it can be concluded that the molecular target of IQ-1 is PR72/130. The interaction of IQ-1 with PR72/130 results in the disruption of the PR72/130/Nkd complex, and inhibits “negative” regulation of canonical Wnt/β-catenin signaling and a concurrent switch to “non-canonical” convergent extension.

### Example 5

### IQ-1 Maintenance of ESCs is WNT/β-catenin/CBP Dependent

Wnt/β-catenin signaling has been demonstrated to inhibit neuronal differentiation and maintain pluripotency in stem cells (1-3) and is critical for the expansion of progenitors (4). However, Wnt/β-catenin signaling is also required for neural differentiation of ESCs and neural stem cells (5-6). Using ICG-001, a recently characterized specific antagonist of the β-catenin/CBP interaction, a model was developed to explain the divergent activities of Wnt/β-catenin signaling (7, 8, 26, 27). The key feature of this model is that β-catenin/CBP-mediated transcription is critical for “stem/progenitor” cell proliferation, whereas a switch to β-catenin/p300-mediated transcription is critical to initiate a differentiative program with a more limited proliferative capacity.

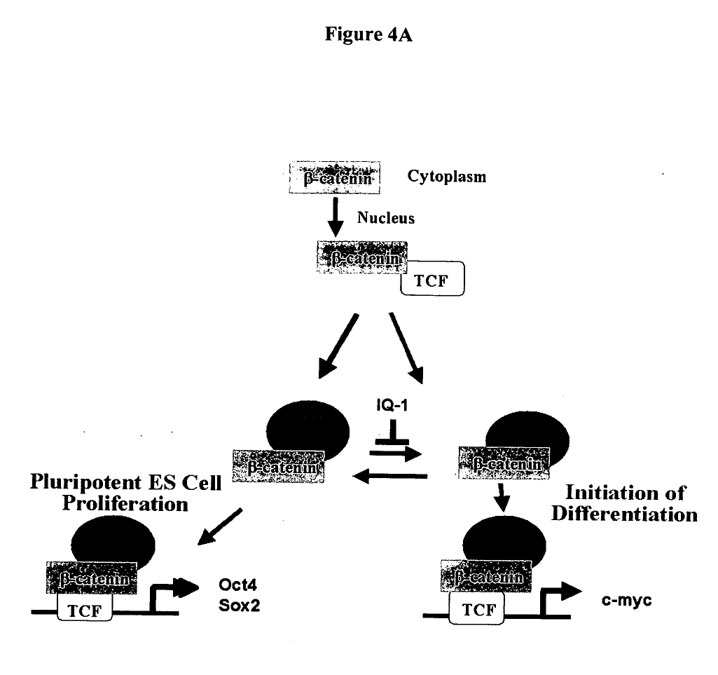

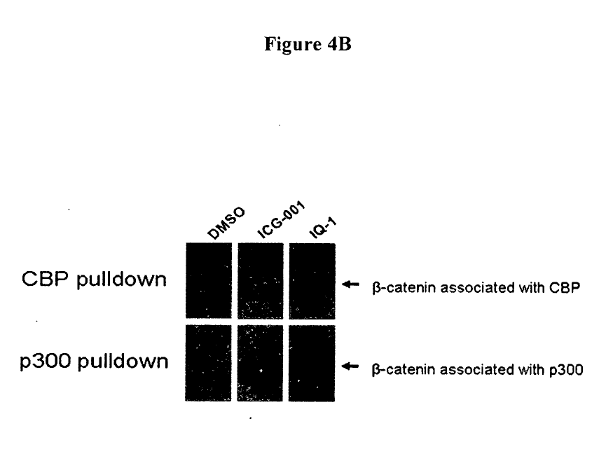

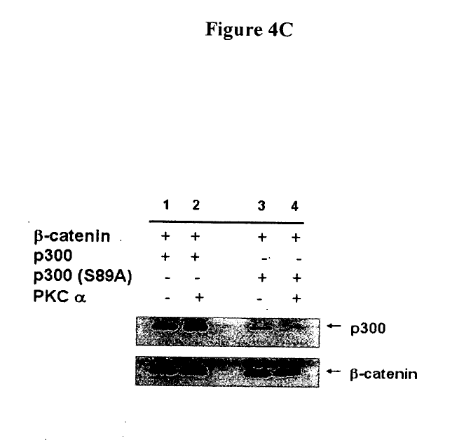

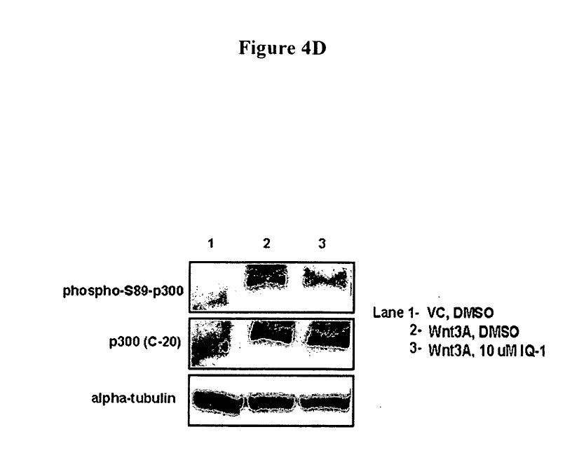

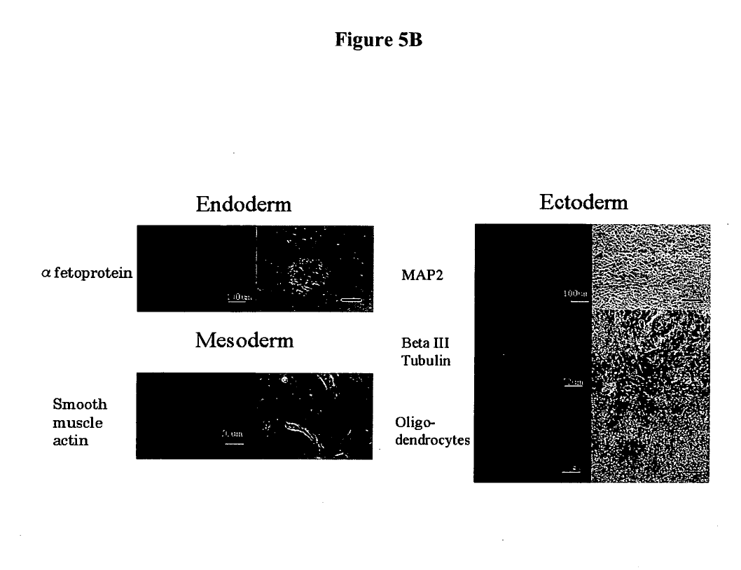

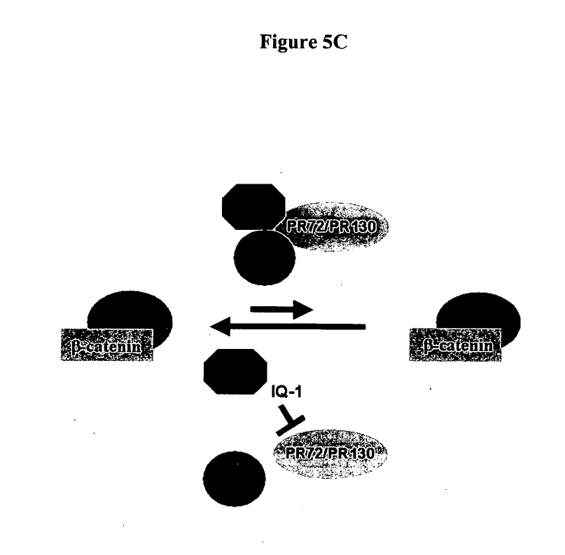

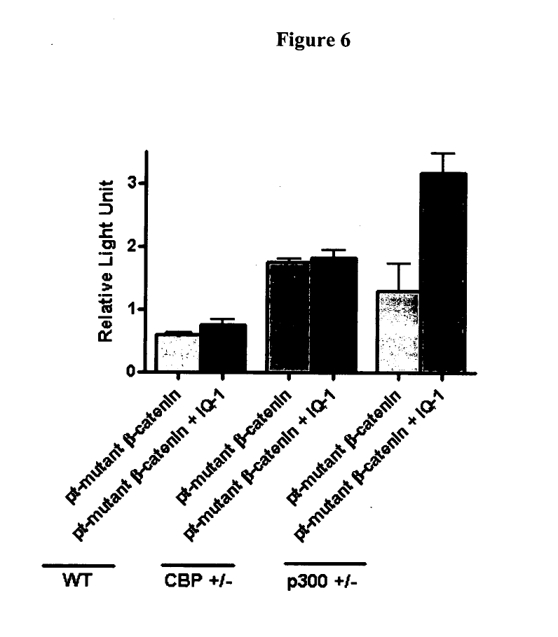

The Wnt/β-catenin reporter constructs TOPFLASH and FOPFLASH in wild-type NIH-3T3 cells (wt), NIH-3T3 CBP(+/−) cells and NIH-3T3 p300(+/−) cells were used to determine whether IQ-1, by targeting the PR72/130 subunit with PP2A, was selectively increasing β-catenin's usage of CBP as a coactivator at the expense of p300, thereby maintaining the ESCs in the undifferentiated state (FIG. 4A). A point mutant constitutively translocating β-catenin (pt-mut β-cat) was used to stimulate reporter activity (28). Although IQ-1 did not affect TOPFLASH or FOPFLASH activity in either the wt or CBP (+/−) cells, a 2-3 fold increase in TOPFLASH activity was observed in the p300 (+/−) cells (FIG. 6). This suggests that the effects of IQ-1 are coactivator specific and dependent on the expression level of p300.

To directly evaluate the effects of IQ-1 on β-catenin coactivator usage, co-immunoprecipitation with antibodies to either CBP or p300 was performed, followed by immunoblotting for coactivator-associated β-catenin utilizing P19 embryonic carcinoma cells treated with Wnt3a in the presence or absence of IQ-1. IQ-1 caused a dramatic increase in the relative amount of β-catenin associated with CBP compared to cells treated with Wnt3a and either DMSO or the CBP/β-catenin antagonist ICG-001. ICG-001, which induces cellular differentiation (7), significantly enhanced the p300/β-catenin interaction at the expense of the CBP/β-catenin interaction (FIG. 4B).

### Example 6

### IQ-1 Indirectly Decreases the Phosphorylation of P300 SER89 and Thereby the β-catenin/P300 Interaction

It is known that signaling through the “non-canonical” Wnt pathway can increase PKC activity (29) and that Ser89 of p300 can be phosphorylated in a PKC-dependent fashion (30). The present example was performed to evaluate the effects of PKCα phosphorylation of p300 Ser89 on the binding of p300 to β-catenin. Recombinant p300 (1-110aa) was phosphorylated with purified PKCα. The in vitro phosphorylated p300 was then added to cell lysates from P19 cells and the β-catenin/p300 complexes were co-immunoprecipitated. Prior phosphorylation by PKCα enhanced the p300/β-catenin interaction (FIG. 4C, compare lane 2 to lane 1). To determine if the enhanced interaction was dependent on p300 Ser89 phosphorylation by PKCα, the serine residue was mutated to alanine in the recombinant p300 fragment. Mutagenesis of Ser89 to Ala89 abrogated the PKCα-dependent increase in binding to β-catenin (FIG. 4C, compare the difference between lanes 3 and 4 to lanes 1 and 2). This indicates that phosphorylation of p300 Ser89 enhances the affinity of the β-catenin/p300 interaction.

To determine the physiological relevance of this phosphorylation-dependent mechanism for increasing the β-catenin/p300 interaction in cells, P19 cells were exposed to purified Wnt3a and either treated with IQ-1 or DMSO control. As judged by immunoblotting, IQ-1 treatment of Wnt3a stimulated P19 cells caused a dramatic decrease in the phosphorylation level of p300 Ser89 (FIG. 4D Top, compare lanes 2 and 3) whereas the total amount of p300 was not affected by IQ-1 (FIG. 4D Middle, compare lanes 2 and 3). Based on these results, it can be concluded that IQ-1 by negatively regulating the phosphorylation of p300 Ser89 thereby decreases the affinity of the β-catenin/p300 interaction and increases β-catenin/CBP usage.

### Example 7

### Long Term Maintenance of ESCs 1N Serum Free Media Containing IQ-1 and WNT3A

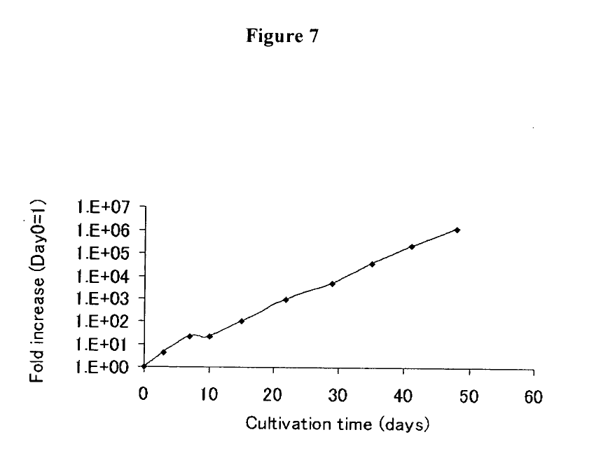

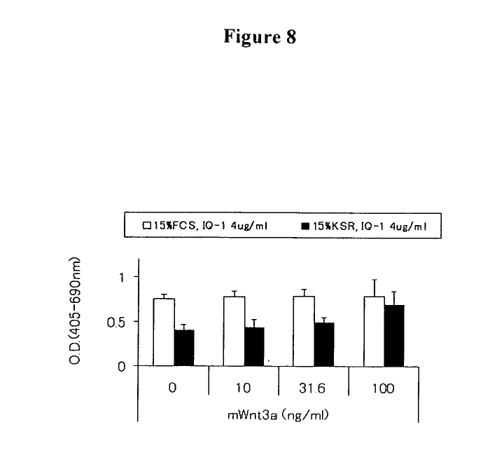

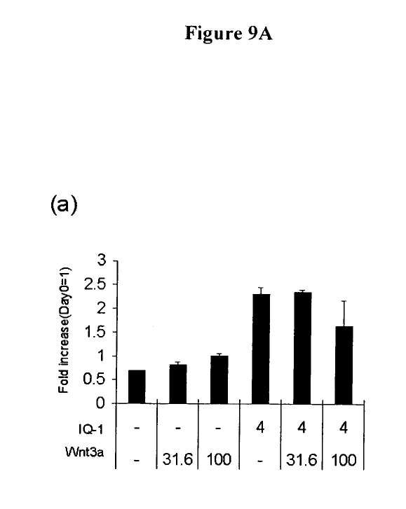

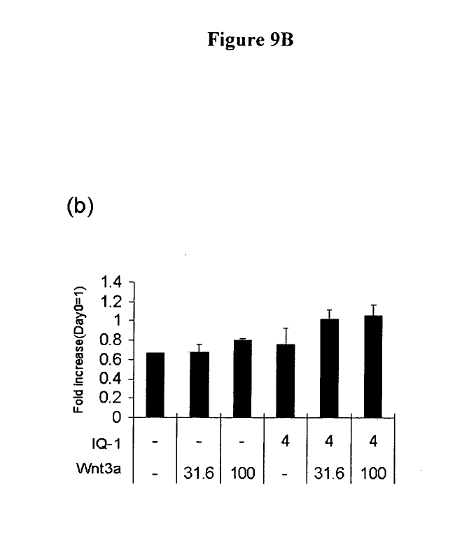

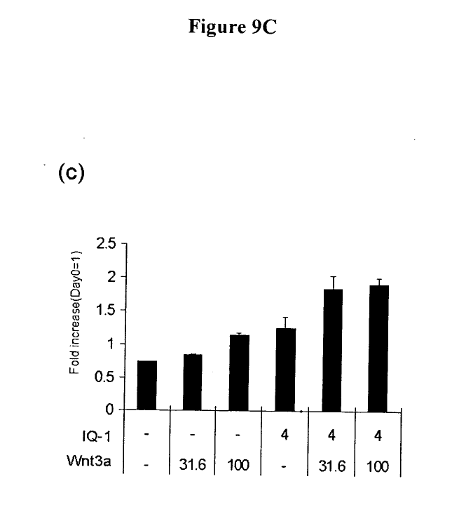

Real-time RT-PCR of P19 cells treated with Wnt3a and IQ-1 revealed increased expression of “stem/progenitor” markers including Oct4, Sox2 (31) and MDR-1 (32) and a decrease in c-myc (33) expression compared to Wnt3a+DMSO treated cells (Table 1). Addition of purified Wnt3a (100 ng/ml) to 15% KSR (serum replacement media) in conjunction with 4 μg/ml of IQ-1 was sufficient to increase alkaline phosphatase levels and maintain ESC pluripotency long term (48 days), similar to results obtained with IQ-1 and 15% FCS (FIG. 7). Neither Wnt3a nor IQ-1 alone was sufficient to maintain the undifferentiated status of ESCs in KSR media (FIG. 8). Wnt3a alone induced proliferation, but was not sufficient to maintain the expression of the stem cell markers Oct4, Nanog or Rex1. However, the combination of Wnt3a and IQ-1 allowed for proliferation and maintenance of pluripotency as judged by the expression of Oct4, Nanog and Rex1 (FIGS. 9A, B, and C). Treatment of ESCs with IQ-1 and Wnt3a maintained the pluripotency of ESCs as judged by their ability to form embryoid bodies after day 48 (FIG. 5A, left panel) and the ability to differentiate to all three germ layer-derived tissues (FIG. 5B). Removal of IQ-1 led to a rapid (within 3 days) loss of pluripotency and the inability to form embryoid bodies (FIG. 5A, right panel). Based on this example, it can be concluded that IQ-1 by increasing Wnt/β-catenin/CBP-dependent signaling and preventing the switch to Wnt/β-catenin/p300 mediated transcription was sufficient to maintain long term murine ESC pluripotency.

TABLE 1

| | ΔΔCT IQ-1 |
|---|---|
| Sox-2 | 4.31 |
| MDR-1 | 1.69 |
| Oct4 | 0.39 |
| c-myc | −2.01 |

In Table 1, above, the effects of IQ-1 treatment on genes associates with stem cell maintenance are shown, as measured by RT-PCR. The values are represented as ΔΔCT from the control gene β-actin.

These Examples demonstrate that IQ-1 in conjunction with purified Wnt3a is sufficient to maintain murine ESC proliferation and pluripotency for extended periods of time in culture (at least 48 days) in the absence of serum. IQ-1 plus Wnt3a upregulated the expression of the transcription factors Oct4 and Sox2, which are critical to ESC maintenance. Recently, Boyer et al. demonstrated that Oct4 and Sox2 co-occupy the promoters/enhancers of a substantial portion of the genes required for the maintenance of human ESCs (31). As shown in the Examples, IQ-1 downregulates the expression of c-myc. C-myc appears to be a critical player in the balance between stem cell self-renewal and differentiation and is increased upon differentiation (33).

Using an affinity chromatography approach, it was determined that the molecular target(s) of IQ-1 are the differentially spliced regulatory subunits PR72/PR130 of the protein phosphatase PP2A. This is extremely interesting in that PR72 interacts with the protein Nkd (25), a Wnt-inducible antagonist of “canonical” Wnt/β-catenin signaling (24). Similar to the results observed with PR72 morpholinos (25), IQ-1 had dramatic effects on “non-canonical” convergent extension during zebrafish embryonic development. The question of the roles of the two splice variants PR72 and PR130 in the switch from “canonical” to “non-canonical” wnt signaling appears to be quite complex. Recently, Bernards et al. showed that unlike PR72, PR130 can antagonize some of the effects of Nkd in a variety of assays. However, PR130 morphilinos affected somite development and tail formation in xenopus embryos (34). Therefore, the effects of IQ-1 on zebrafish embryonic development are consistent with potentially inhibiting both splice variants. Without being bound by a particular mechanism, the results are consistent with a model in which the interaction of IQ-1 with PR72/130 results in the disruption of the PR72/130 Nkd complex thereby modulating Wnt signaling. The morphological effects on zebrafish development are consistent with a model wherein IQ-1 inhibits the “negative” regulation of canonical Wnt/β-catenin signaling and a concurrent switch to “non-canonical” convergent extension (FIG. 5C).

In vitro phosphorylation of recombinant p300 by PKC, an enzyme activated via “non-canonical” Wnt signaling, increased the affinity of the β-catenin for p300. Furthermore in cells, IQ-1 significantly decreased Wnt-stimulated phosphorylation of p300 at Ser89, without affecting the overall cellular level of p300. The mechanism by which IQ-1 can decrease the phosphorylation status of p300 at Ser89 remains unclear and is the subject of ongoing investigations.

IQ-1 selectively promoted the β-catenin/CBP interaction at the expense of the corresponding β-catenin/p300 interaction. IQ-1 by enhancing Wnt/β-catenin/CBP-mediated transcription and preventing the switch to Wnt/β-catenin/p300-mediated transcription allows for the long term expansion of murine ESCs while maintaining pluripotency without MEFs or serum.

Differential coactivator usage in Wnt/β-catenin signaling appears to be a critical regulator in the maintenance of the “stem/progenitor” state, the initiation of differentiation with a more restricted proliferative capacity, as well as the switch from “canonical” to “non-canonical” Wnt signaling. IQ-1 's effects on Wnt-mediated pluripotency are associated with inhibition of the “negative” regulation of canonical Wnt/β-catenin signaling by the Nkd/PR72/PP2A complex, thereby increasing β-catenin/CBP-driven transcription at the expense of β-catenin/p300-driven transcription.

The ability to expand “stem/progenitor” populations under defined growth conditions has important ramifications in the area of regenerative medicine, and the Examples herein support such use.

### REFERENCES

1 Willert, K., Brown, J. D., Danenberg, E., Duncan, A. W., Weissman, I. L., Reya, T., Yates, J R 3^{rd} & Nusse, R. (2003) Nature 423, 448-452.
2 Haegele, L., Ingold, B., Naumann, H., Tabatabai, G., Ledermann, B. & Brandner, S. (2003) Mol. Cell. Neurosci. 24, 696-708.
3 Sato, N., Meijer, L., Skaltsounis, L., Greengard, P. & Brivanlou, A. (2004) Nat. Med. 10, 55-63.
4 Chem, A. & Walsh, C. A. (2002) Science 297, 365-369.
5 Otero, J. J., Fu, W., Kan, L., Cuadra, A. E. & Kessler, J. A. (2004) Development 131, 3545-3557(2004).
6 Muroyama, Y., Kondoh, H. & Takada, S. (2004) Biochem. Biophys. Res. Commun. 313, 915-921.
7 Teo, J. L., Ma, H., Nguyen, C., Lam, C. & Kahn, M. (2005) Proc. Natl. Acad. Sci. U.S.A. 102, 12171-12176.
8 McMillan, M. & Kahn, M. (2005) Drug Discov. Today 10, 1467-1474.
9 Xu, C., Inokuma, M. S., Denham, J., Golds, K., Kundu, P., Gold, J. D., & Carpenter, M. (2001) Nat. Biotech. 19, 971-974.
10 Lu, J., Hou, R., Booth, C. J., Shih-Hung Yang, S and Snyder, M. (2006) Proc Natl Acad Sci U.S.A. 103, 5688-5693.
11 Yao, S., Chen, S., Clark, J., Hao, E., Beattie, G. M., Hayek, A. & Ding, S. (2006) Proc Natl Acad Sci U.S.A. 103, 6907-6912.
12 Pease, S., Braghetta, P., Gearing, D., Grail, D. & Williams, R L. (1990) Dev. Biol. 141, 344-352.
13 Smith, A. G., Heath, J. K., Donaldson, D. D., Wong, G. G., Moreau, J., Stahl, M. & Rogers, D. (1988) Nature 336, 688-690.
14 Williams, R. L., Hilton, D. J., Pease, S., Willson, T. A., Stewart, C. L., Gearing, D. P., Wagner, E. F., Metcalf, D., Nicola, N. A. & Gough, N. M. (1988) Nature 336, 684-687.
15 Niwa, H., Burdon, T., Chambers, I., & Smith, A. (1998) Genes Dev. 12, 2048-2060.
16 Humphrey, R. K., Beattie, G. M., Lopez, A. D., Bucay, N., King, C. C., Firpo, M. T., Rose-John, S. & Hayek, A. (2004) Stem Cells 22, 522-530.
17 Daheron, L., Opitz, S. L., Zaehres, H., Lensch, W. M., Andrews, P. W., Itskovitz-Eldor, J. & Daley, G. Q. (2004) Stem Cells 22, 770-778.
18 Mitsui, K., Tokuzawa, Y., Itoh, H., Segawa, K., Murakami, M., Takahashi, K., Maruyama, M., Maeda, M. & Yamanaka, S. (2003) Cell 113, 631-642.
19 Chambers, I., Colby, D., Robertson, M., Nichols, J., Lee, S., Tweedie, S. & Smith, A. (2003) Cell 113, 643-655.
20 Mumby, M. C. & Waler, G. (1993) Physiol. Rev. 73, 673-699.
21 Hendrix, P., Mayer-Jackel, R. E., Cron, P., Goris, J., Hofsteenge, J., Merlevede, W., Hemmings, B. A. (1993) J. Biol. Chem. 268, 15267-15276.
22 Seeling, J. M., Miller, J. R., Gil, R., Moon, R. T., White, R. & Virshup, D. M. (1999) Science 283, 2089-2091.
23 Ratcliffe, M. J., Itoh, K. & Sokol, S. Y. (2000) J. Biol. Chem. 275, 35680-35683.
24 Zeng, W., Wharton, K. A. Jr, Mack, J. A., Wang, K., Gadbaw, M., Suyama, K., Klein, P. S., Scott, M. P. (2000) Nature 403, 789-795.
25 Creyghton, M. P., Roël, G., Eichhorn, P. J. A., Hijmans, E. M., Maurer, I., Destrée, O. & Bernards, R. (2005) Genes. Dev. 19, 376-386.
26 Emami, K. H., Nguyen, C., Ma, H., Kim, D. H., Jeong, K. W., Eguchi, M., Moon, R. T., Teo, J. L., Kim, H. Y., Moon, S. H., et al. (2004) Proc. Natl. Acad. Sci. U. S. A. 101, 12682-12687.
27 Ma, H., Nguyen, C., Lee, K. S. & Kahn, M. (2005) Oncogene 24, 3619-3631.
28 Korinek, V., Barker, N., Morin, P. J., van Wichen, D., de Weger, R., Kinzler, K. W., Vogelstein, B. & Clevers, H. (1997) Science 275, 1784-1787.
29 Kohn, A. D. & Moon, R. T. (2005) Cell Calcium 38, 439-446.
30 Yuan, L. W. & Gambee, J. E. (2000) J. Biol. Chem. 275, 40946-40951.
31 Boyer, L. A., Lee, T. I., Cole, M. F., Johnstone, S. E., Levine, S. S., Zucker, J. P., Guenther, M. G., Kumar, R. M., Murray, H. L., Jenner, R. G. et al. (2005) Cell 122, 947-956.
32 Feuring-Buske, M. & Hogge, D. E. (2001) Blood 97, 3882-3889.
33 Wilson, A., Murphy, M. J., Oskarsson, T., Kaloulis, K., Bettess, M. D., Oser, G. M., Pasche, A. C., Knabenhans, C., Macdonald, H. R. & Trumpp, A. (2004) Genes Dev 18, 2747-2763.
34 Creyghton, M. P., Roel, G., Eichorn, P. J. A., Vredeveld, L. C., Destree, O. & Bernards, R. (2006) Proc. Natl. Acad. Sci. U. S. A. 103, 5397-5402.
35 Zandstra, P. W., Le, H. V., Daley, G. Q., Griffith, L. G. & Lauffenburger, D. A. (2000) Biotechnol Bioeng. 69, 607-617.

The foregoing specification, including the specific embodiments and examples, is intended to be illustrative of the present invention and is not to be taken as limiting. Numerous other variations and modifications can be effected without departing from the true spirit and scope of the invention. All patents, patent applications, provisional applications, and publications referred to or cited herein are incorporated by reference in their entirety, including all figures and tables, to the extent they are not inconsistent with the explicit teachings of this specification.

1. A method of identifying an agent capable of maintaining proliferation of a mammalian stem cell, said method comprising contacting an agent with at least one of subunits PR72/130 of the serine/threonine protein phosphatase PP2A and detecting binding of said agent to said at least one subunit.

2. The method of claim 1, wherein said binding is detected by immunoblotting.

3. The method of claim 1, wherein said agent is selected from the group consisting of compounds of formula 1-5, 7-12, 15 and 17-28.

4. A method of enhancing the proliferation of a mammalian stem cell, comprising administering to said stem cell an agent that selectively modulates the interaction of β-catenin with CBP or p300.

5. The method of claim 4, wherein the agent increases the binding of β-catenin to CBP.

6. (canceled)

7. The method of claim 4, wherein said administration to said stem cells is ex vivo.

8. The method of claim 4, wherein said stem cells are hematopoietic stem cells.

9. (canceled)

10. The method of claim 4, wherein said stem cells are neural stem cells.

11. The method of claim 4, wherein said stem cells are pancreatic islet cells.

12. (canceled)

13. The method of claim 4, wherein said stem cells are muscle stem cells.

14. The method of claim 4 whereby the compound interacts with either one of the differentially spliced regulatory subunits PR72/130 of the serine/threonine protein phosphatase PP2A and increases the interaction of β-catenin/CBP at the expense of the β-catenin/p300 interaction.

15. The method of claim 4 whereby the compound in conjunction with canonical Wnt stimulation increases the expression of Oct4 and Sox2 and decreases the expression of c-myc.

16. The method of claim 15 wherein the compound in conjunction with purified Wnt3a increases the expression of Oct4 and Sox2 and decreases the expression of c-myc.

17. The method of claim 15 wherein the compound in conjunction with inhibition of GSK-3 activity increases the expression of Oct4 and Sox2 and decreases the expression of c-myc.

18. The method of claim 14 whereby the compound causes the proliferation without differentiation of a stem cell in conjunction with canonical Wnt stimulation.

19. (canceled)

20. The method of claim 18 wherein said stem cell in an adult stem cell.

21. A composition for enhancing the proliferation of a mammalian stem cell, comprising administering to said stem cell a compound having the biological activity of IQ-1.

22. The composition of claim 21 wherein said biological activity is selected from the group consisting of preventing phosphorylation of p300, maintaining expression of Nanog in embryonic stem cell culture, maintaining expression of Oct3/4 in ESC culture and maintaining expression of Rex-1 in ESC culture.

23. The composition of claim 21, wherein said compound is IQ-1.

24. The method of claim 4 whereby the compound(s) inhibit the MAPK kinase pathway (MEK or ERK) and PKC in conjunction with stimulation of the canonical Wnt pathway, either with Wnt3a or a GSK3 inhibitor.
